SEQUENCE LISTING (2 sequences)

SEQ ID NO: 1 - DNA, 24 bp, artificial
  Synthetic primer
tcacacctgtaatcccagcacttt

SEQ ID NO: 2 - DNA, 19 bp, artificial
  Synthetic primer
gcccaggctggagtgcagt

(19) United States
(12) Patent Application Publication
(10) Pub. No.: US 2017/0242015 A1
(43) Pub. Date: Aug. 24, 2017

(54) MAP3K8 AS A MARKER FOR SELECTING A PATIENT AFFECTED WITH AN OVARIAN CANCER FOR A TREATMENT WITH A MEK INHIBITOR

(71) Applicants: INSTITUT CURIE, Paris (FR); INSERM (INSTITUT NATIONAL DE LA SANTÉ ET DE LA RECHERCHE MÉDICALE), Paris (FR)

(72) Inventors: Fatima MECHTA-GRIGORIOU, Paris (FR); Virginie MIEULET, Paris (FR)

(21) Appl. No.: 15/510,848

(22) PCT Filed: Sep. 15, 2015
(86) PCT No.: PCT/EP15/71040

§ 371 Date: Mar. 13, 2017

(30) Foreign Application Priority Data
Sep. 17, 2014 (EP) 14306434.3

(51) Int. Cl. G01N 33/574; A61K 31/4184; C12N 15/113

(52) U.S. Cl. CPC G01N 33/57449; C12N 15/1137; A61K 31/4184; G01N 2333/912; C12N 2310/11; C12N 2310/14; C12N 2310/531

(57) ABSTRACT

The present invention relates to the use of MAP3K8 protein to predict clinical outcome of a patient affected with a cancer, in particular ovarian cancer, or to select a patient for a treatment comprising one MEK inhibitor. The invention further relates to a method for monitoring a patient affected with a high-MAP3K8 ovarian cancer (i.e. an ovarian cancer with a high expression and/or activity level of MAP3K8) to a treatment comprising at least one MEK inhibitor. The invention also further relates to a MEK inhibitor for use in the treatment of high-MAP3K8 ovarian cancer and kit that can be used in the methods of the invention.

### FIELD OF THE INVENTION

The present invention relates to the field of medicine, in particular of oncology. It relates to a new marker (i.e. MAP3K8) to classify patients suffering from an ovarian cancer for a treatment comprising at least one MEK inhibitor.

### BACKGROUND OF THE INVENTION

Cancer occurs when cell division gets out of control and may result from impairment of a DNA repair pathway, the transformation of a normal gene into an oncogene and/or the malfunction of a tumor suppressor gene. Many different forms of cancer exist. The incidence of these cancers varies but overall, cancer is the second highest cause of mortality, after heart disease, in most developed countries. While different forms of cancer have different properties, one factor which many cancers share is the ability to metastasize. Distant metastasis of all malignant tumors remains the primary cause of death in patients with the disease.

Ovarian cancer, especially epithelial ovarian cancer (EOC), is the most common form of gynaecologic malignancies and the fifth most frequent cause of cancer death in women (Yancik, R., cancer 1993). Because of the insidious onset of the disease and the lack of reliable screening tests, patients are often diagnosed with advanced disease. The established prognostic factors in ovarian cancer are based on age, stage, histology, grade, volume of ascites, performance status, extent of residual disease following debulking surgery and findings at second-look laparotomy (Berman M L., Gynecol Oncol 2003; Batista L et al. Int J Biochem Cell Biol 2013). Although many patients respond initially to standard combinations of surgery and chemotherapy, most of them will develop cancer recurrence and eventually succumb to their disease.

In recent years, several studies have analyzed large-scale transcription profiling to identify differentially expressed genes in ovarian cancer according to tumor status, histological subtypes and metastatic spread. Yet, the molecular biology of ovarian cancer is still not completely understood, making difficult the development of more effective therapies.

Ovarian cancers may be of different sub-types with different pathological features and outcomes. Due to these variations, the appropriate therapy for each of this sub-type may differ.

Considerable efforts have been made in order to find markers that could be used to classify ovarian cancers and allowing a reliable patient stratification for a more adapted therapeutic strategy.

In this aim, the potential use of microRNA, transcriptomic and proteomic data has been extensively studied. For example, miR-200 family members have been shown to accumulate in ovarian cancer (Iorio M. V et al., Cancer Res 2007; Nam E. J et al., Clin Cancer Res 2008; Hu X et al., Gynecol Oncol 2009; Bendoraite A et al., Gynecol Oncol 2010). However, the correlation between the expression of miR-200s and of ovarian cancer prognosis remains uncertain. Indeed, in these studies, it has been shown that high expression of miR-200 could be linked to poor prognosis (Nam E. J et al., Clin Cancer Res 2008) or to good prognosis (Hu X et al., Gynecol Oncol 2009).

Recently, transcriptomic and proteomic analysis have allowed identifying two subgroups of patients affected with ovarian cancer, based on the presence of an “Oxidative stress” signature or of a “Fibrosis” signature (Mateescu et al., Nature Med 2011; Batista L et al., Int J Biochem Cell Biol 2013). WO2012072846 describes that the “Oxidative stress” group characterized by a high expression level of GSR and a low expression level of MYL9 is associated with a better global prognosis than the “Fibrosis” group and then can be better treated with a treatment comprising an antineoplastic agent inducing the accumulation of ROS.

However, there is still a strong need to provide new reliable markers for stratifying patients for therapeutic intervention, for identifying relevant treatment and for monitoring the response to said treatment in ovarian cancer patients and especially EOC.

### SUMMARY OF THE INVENTION

In the present invention, the inventors demonstrate that MAP3K8 is a prognostic marker in human ovarian cancers. They indeed show that an ovarian cancer with a high expression level and/or activity level of MAP3K8 is of poor prognosis as it is associated with marked decrease in patient's progression free survival as well as overall survival. They also show that MAP3K8 controls proliferation, migration, invasion of EOC and tumor growth by activating the MEK/ERK pathway. Therefore MAP3K8 is a predictive marker for treatment with at least one MEK inhibitor in ovarian cancer.

A first aspect of the present invention is the use of MAP3K8 as a marker.

In one embodiment, the present invention relates to the use of MAP3K8 as a prognostic marker in ovarian cancer.

In a second embodiment, the present invention relates to the use of MAP3K8 as a marker for selecting a subject affected with an ovarian cancer for a treatment comprising at least one MEK inhibitor, or determining whether a subject affected with an ovarian cancer is susceptible to benefit from a treatment comprising at least one MEK inhibitor, preferably comprising the step of determining the expression level and/or the activity level of MAP3K8, and wherein a high expression level and/or a high activity level of MAP3K8 being indicative that said subject is susceptible to benefit from said treatment.

A second aspect of the present invention relates to methods of prognosis, patient selection and treatment monitoring using MAP3K8 as a marker.

In one embodiment, the present invention relates to a method for predicting the clinical outcome of a subject affected with an ovarian cancer, wherein the method comprises the step of determining the expression level and/or the activity level of MAP3K8 in a cancer sample from said subject, a high expression level and/or a high activity level of MAP3K8 being indicative of a poor prognostic.

In a second embodiment, the present invention relates to a method for selecting a subject affected with an ovarian cancer for a treatment comprising at least one MEK inhibitor, or determining whether a subject affected with an ovarian cancer is susceptible to benefit from a treatment comprising at least one MEK inhibitor, wherein the method comprises the step of determining the expression level and/or the activity level of MAP3K8 in a cancer sample from said subject, a high expression level and/or a high activity level of MAP3K8 being indicative that said subject is susceptible to benefit from said treatment.

In a further embodiment, the present invention relates to a method for monitoring the response of a subject affected with an ovarian cancer with a high expression level and/or activity level of MAP3K8, to a treatment comprising at least one MEK inhibitor, wherein the method comprises the step of determining P-ERK/ERK ratio in an ovarian cancer sample from said subject, a low P-ERK/ERK ratio being indicative that said subject is responsive to the treatment comprising at least one MEK inhibitor.

In a further embodiment, the present invention relates to a method for monitoring the response of a subject affected with an ovarian cancer with a high expression level and/or a high activity level of MAP3K8 to a treatment comprising at least one MEK inhibitor, wherein the method comprises the step of determining P-ERK/ERK ratio in a cancer sample from said subject, a high P-ERK/ERK ratio being indicative that the subject is not responsive and/or resistant to said treatment comprising at least one MEK inhibitor.

Another aspect of the present invention concerns a kit and its use (a) for predicting clinical outcome of a subject affected with an ovarian cancer, and/or (b) for selecting a subject affected with an ovarian cancer for a treatment comprising at least one MEK inhibitor, or determining whether a subject affected with an ovarian cancer is susceptible to benefit from a treatment comprising at least one MEK inhibitor, wherein the kit comprises means for detecting the expression level and/or activity level of MAP3K8, preferably comprising at least one antibody specific to MAP3K8 and optionally, a leaflet providing guidelines to use such a kit.

Another aspect of the present invention concerns a kit and its use for monitoring the response to a treatment comprising at least one MEK inhibitor of a subject affected with an ovarian cancer, particularly an ovarian cancer with a high expression level and/or activity level of MAP3K8 wherein the kit comprises:
(i) at least one antibody specific to P-ERK or ERK; and/or
(ii) at least one probe specific to the ERK mRNA or cDNA; and/or
(iii) at least one nucleic acid primer pair specific to ERK mRNA or cDNA; and optionally, a leaflet providing guidelines to use such a kit.

The kits of the invention may further comprise means for detecting the formation of complexes between proteins (i.e. MAP3K8 or ERK) and their specific antibodies (i.e. antibodies specific to MAP3K8 or P-MAP3K8 or ERK or P-ERK).

The kit for monitoring the response to the treatment comprising at least one MEK inhibitor may further comprise (i) means for detecting the hybridization of the probes with the ERK mRNA or cDNA; and/or (ii) means for amplifying and/or detecting the ERK mRNA or cDNA molecules by using their pairs of primers.

The kits of the invention can further comprise control reagents and other necessary reagents.

A further aspect of the present invention concerns a MEK inhibitor for use in the treatment of an ovarian cancer with a high expression level and/or a high activity level of MAP3K8.

A further aspect of the present invention relates to a MEK inhibitor for use in a treatment for reducing and/or preventing lung metastatic incidence related to ovarian cancer.

In an embodiment, said MEK inhibitor is selected from the group consisting of a small molecule, an antibody, a nucleic acid, an aptamer, a peptide, a polypeptide, a protein or any molecule preventing the interaction of MEK with a MEK interacting partner (such as ERK or MAP3K8).

In another embodiment, said MEK inhibitor is selected from the group consisting of a small molecule, an antibody against MEK and a nucleic acid molecule interfering specifically with MEK expression such as antisense against MEK, a siRNA against MEK and a shRNA against MEK. Preferably, said MEK inhibitor is a small molecule.

In a further embodiment, said MEK inhibitor is a small molecule selected from the group consisting of artigenin, AS703026, AZD8330, AZD6244 (Selumetinib), BAY 869766, KT 5720, AS-252424, BIX 02189, Debromohymenialdisine, Hypothemycin, MEK Inhibiteur II, PD 0325901, PD 184,352, SB 203580, PD 184161, PD 198306, PD 98059, PD 318088, Selumetinib, SL-327, TAK-733, Trametinib, U-0126, U-0124, 2-Bromoaldisine, Myricetin, Chk2 Inhibiteur, Honokiol, cobimetinib, XL518, CI-1040 and MEK162.

In an even further embodiment, said MEK inhibitor is selected from the group consisting of PD 0325901, AZD6244 and MEK162.

In another embodiment, said MEK inhibitor is a nucleic acid molecule interfering specifically with MEK expression or with the interaction between MEK with one of their specific partners and is selected from the group consisting of an antisense against MEK, a siRNA against MEK and a shRNA against MEK.

In an aspect, said MEK inhibitor is selected from the group consisting of an antibody against MEK and a nucleic acid molecule interfering specifically with MEK expression

In another embodiment, said MEK inhibitor is a molecule preventing the interaction of MEK with a MEK interacting partner (such as ERK) and is selected from the group consisting of an aptamer, an antibody, a peptide, a polypeptide and a protein.

In a further aspect, the present invention concerns the use of the above-mentioned MEK inhibitors in combination with any other type of treatment suitable to treat ovarian cancer, including surgery, radiotherapy or a chemotherapeutic agent.

In another embodiment, the present invention relates to i) a method for treating an ovarian cancer, preferably an ovarian cancer with a high level and/or a high activity level of MAP3K8, by administering a therapeutic effective amount of a MEK inhibitor and ii) to the use of a MEK inhibitor for treating an ovarian cancer, preferably an ovarian cancer with a high level and/or a high activity level of MAP3K8.

In still another embodiment, the ovarian cancer is an ovarian cancer with a high expression level and/or a high activity level of MAP3K8. Particularly, the ovarian cancer is an EOC, more preferably a high-grade and/or advanced-stage EOC.

In still another embodiment, the methods are in vivo, ex vivo and in-vitro methods, preferably in-vitro methods.

### BRIEF DESCRIPTION OF THE DRAWINGS

FIG. 1: MAP3K8 is a prognostic marker in human ovarian carcinomas: (a) Kaplan-Meier curves showing progression free survival (PFS) (left panel) and overall survival (OS) (right panel) of EOC patients, with respect to low-MAP3K8 (N=24 patients) or high-MAP3K8 (N=48 patients) protein levels. P-values are based on Log-rank test. (b) Left: Box plots showing MAP3K8 protein levels, as assessed by densitometry analysis of western blots (representative blot shown in FIG. 7 (a)) in “Stress” (N=32) and “Fibrosis” (N=40) patients. Middle: Box plot showing two distinct sub-groups of “Stress” patients, according to MAP3K8 protein level. Right: Bar plot showing a significant association between MAP3K8 protein levels and the “Stress” (N=32) and “Fibrosis” (N=40) subgroups of patients. P-values are based on Welch's t-test (Left and Middle panels) and Fisher's exact test (Right panel). (c) Kaplan-Meier curves showing PFS (left panel) and OS (right panel) of EOC patients from the “Stress” or “Fibrosis” subgroup, with respect to MAP3K8 protein levels. P-values are based on Log-rank test. (d) Kaplan-Meier curves showing PFS (left panel) and OS (right panel) of EOC patients, with respect to low- (N=24) or to high- (N=34) MAP3K8 protein levels. The subgroups of EOC characterized by high-MAP3K8 protein levels are either Immunoreactive (N=13) or Mesenchymal (N=21), regarding the DIMP signature. P-values are based on Log-rank test (N=58 tumors). ns stand for “not significant” and N refers to the number of patients. Scale bars: 50 μm.

FIG. 2: MAP3K8 controls proliferation, migration and invasion of ovarian cancer cells and tumor growth in mouse xenograft models: (a) Representative views of MAP3K8 immunostaining from human ovarian tumors that exhibit low-MAP3K8 (top panel) or high-MAP3K8 (bottom panel) protein levels. Scale bars: 50 μm. (b) Top: Western blot analysis of MAP3K8 protein level in SKOV3 stable cell lines expressing non-targeting shRNA (shCtrl) or two different MAP3K8-targeting shRNA (shMAP3K8_1 and shMAP3K8_2). GAPDH is used as an internal control for protein loading. Bottom: Bar plot showing the ratio of MAP3K8/GAPDH protein levels as assessed by densitometry analysis of western blots (as shown above) obtained from independent experiments and expressed as percentage of shCtrl. P-values are based on one sample t-test. Data are shown as means±s.e.m (n=5 independent experiments). (c) Growth curve of SKOV3 stable cell lines (shCtrl, shMAP3K8_1 and shMAP3K8_2), for the indicated times. P-values are based on Paired t-test. * stands for p-value≦0.05 and **, p-value≦0.005. Data are shown as means±s.e.m (n=5 independent experiments). (d) Growth curve of SKOV3 cells either left untreated (Ctrl) or treated with 5 μM MAP3K8 kinase inhibitor (KI), for the indicated times. P-values are based on Student's t-test. * stands for p-value≦0.05; **, p-value≦0.005 and ***, p-value≦0.0005. Data are shown as means±s.e.m (n=3 independent experiments). (e) Bar plot shows doubling time of SKOV3 cells either left untreated (Ctrl) or treated with MAP3K8 kinase inhibitor (KI). P-value is based on Welch's t-test. Data are shown as means±s.e.m (n=3 independent experiments). (f) Bar plot represents the percentage of SKOV3 cells, in G1, S and G2 phases of the cell cycle, in untreated (Ctrl) cells or following treatment with MAP3K8 kinase inhibitor (KI), for the indicated times. P-values are based on Welch's t-test. * stands for p-value≦0.05. Data are shown as means±s.e.m (n=3 independent experiments). (g) Representative cell cycle distribution of SKOV3 cells without (Ctrl) or with KI treatment, at 12 hours time point. (h) Bar plots represent cell migration (left panel) or invasion (right panel) of SKOV3 stable cell lines (shCtrl, shMAP3K8_1 and shMAP3K8_2) as percentage of shCtrl. P-values are based on one-sample t-test. Data are shown as means±s.e.m (n≧3 independent experiments). (i) Bar plots represent cell migration (left panel) or invasion (right panel) of SKOV3 cells either left untreated (Ctrl) or treated with MAP3K8 kinase inhibitor (KI). P-values are based on one-sample t-test. Data are shown as means±s.e.m (n≧3 independent experiments). (j) Tumor growth curves over time from one representative experiment of xenografted SKOV3 stable cell lines expressing shCtrl (N=12 tumors), shMAP3K8_1 (N=12 tumors) or shMAP3K8_2 (N=10 tumors), as indicated (n=3 independent experiments). P-value are based on Welch's t-test. * stands for p-value≦0.05 and **, p-value≦0.005. Data are shown as means±s.e.m (N≧10 tumors per group).

FIG. 3: MEK/ERK signaling is impaired upon MAP3K8 inhibition: (a) Western blots showing MAP3K8, P-MEK, MEK, P-ERK, ERK, P-JNK, JNK, P-NFκB, NFκB, P-p38, and p38 protein levels in SKOV3 stable cell lines (shCtrl, shMAP3K8_1 and shMAP3K8_2) either without serum (−FBS) or following 15 minutes of serum stimulation (+FBS). GAPDH is used as an internal control for protein loading. (b) Bar graphs showing MAP3K8/GAPDH, P-MEK/MEK, P-ERK/ERK, P-JNK/JNK, P-NFκB/NFκB and P-p38/p38 ratios as indicated, and assessed by densitometry analysis of western blots (as shown in a) obtained from independent experiments and expressed as compared to shCtrl. P-values are based on Welch's t-test. Data are shown as means±s.e.m (n≧5 independent experiments). ns stand for “not significant”. (c) Western blots showing P-MEK, MEK, P-ERK, ERK, P-JNK, JNK, P-NFκB, NFκB, P-p38, and p38 protein levels in SKOV3 cells either without serum (−FBS) or following 15 min of serum stimulation (+FBS), in absence or in the presence of MAP3K8 kinase inhibitor (KI) at 2 different doses, as indicated. (d) Bar plots showing P-MEK/MEK, P-ERK/ERK, P-JNK/JNK, P-NFκB/NFκB and P-p38/p38 ratios, as indicated, and assessed by densitometry analysis of western blots (as shown in c) obtained from independent experiments, and expressed as compared to the serum-stimulated control condition. P-values are based on Welch's t-test. Data are shown as means±s.e.m (n≧5 independent experiments). ns stand for “not significant”.

FIG. 4: MAP3K8 level correlates with its kinase activity and MEK/ERK activation in human EOCs: (a) Western blot showing P-MEK, MEK, P-ERK and ERK protein levels in human EOC classified as low- (N=18 EOC) or high-MAP3K8 (N=36 EOC) protein levels. Actin is used as an internal control for protein loading. (b) Scatter plots of P-MEK/MEK and P-ERK/ERK ratios as indicated, in low- and high-MAP3K8 human EOC. Data were assessed by densitometry analysis of western blots, as shown in (a). P-values are based on Mann-Whitney test. Data are shown as means±s.e.m (N≧18 EOC per group). (c) Scatter plots of P-p38/p38 and P-NFκB/NFκB ratios, as indicated, in low- (N=18) and high-MAP3K8 (N=33) human EOC. Data are from RPPA analysis. P-values are based on Mann-Whitney test. Data are shown as means±s.e.m (N≧18 tumors per group). ns stand for “not significant”. (d) Correlation between P-ERK/ERK and P-MEK/MEK (left panel); MAP3K8/Actin and P-MEK/MEK (middle panel); and MAP3K8/Actin and P-ERK/ERK (right panel) in human EOC. Values have been assessed by densitometry analysis of western blots, as those shown in (a and FIG. 7). Correlation coefficient σ and p-value are based on Spearman's rank correlation test (N=54 EOC). (e) Western blot showing MAP3K8 phosphorylated forms on Threonine 290 (T290) or on Serine 400 (S400), as well as P-MEK and MEK protein levels, before (Input) or after immunoprecipitation (IP:tag) with a Myc-Tag specific antibody performed on protein extracts from SKOV3 cells either transfected with empty vector (Ctrl) or transfected with increasing amounts of myc-tagged MAP3K8 expression vector (tag-MAP3K8). The Myc-tag is used as an internal control for MAP3K8 protein levels (tag). (f) Correlation between P-MAP3K8 (T290) and MAP3K8 protein levels, as assessed by densitometry analysis of western blots (as shown in e) obtained from independent experiments. Correlation coefficient σ and p-value are based on Spearman's rank correlation test (n=4 independent experiments). (g) Correlation between P-MAP3K8 (T290) protein levels and P-MEK/MEK ratios, as assessed by densitometry analysis of western blots (as shown in e) obtained from independent experiments. Correlation coefficient σ and p-value are based on Spearman's rank correlation test (n=4 independent experiments). (h) Western blot showing P-MEK and MEK protein levels in mouse xenografted tumors (N≧6 tumors per group) derived from shCtrl, shMAP3K8_1 or shMAP3K8_2 SKOV3 stable cell lines (tumor growth described above, FIG. 2J). GAPDH is used as an internal control for protein loading. (i) Scatter plot of P-MEK/MEK ratio in mouse xenografted tumors derived from shCtrl, shMAP3K8_1 and shMAP3K8_2 SKOV3 stable cell lines, as assessed by densitometry analysis of the western blot shown in (h). P-values are based on Mann-Whitney test. Data are shown as means±s.e.m (N≧6 tumors per group).

FIG. 5: MAP3K8 is a predictive marker for MEK inhibitor treatments: (a) Western blot showing MAP3K8, P-MEK and MEK protein levels in different tumors derived from 2 patient-derived xenograft (PDX) mouse models characterized either by high- or low-MAP3K8 protein levels, as indicated (N=5 tumors per group). Actin is used as an internal control for protein loading. (b) Scatter plots of MAP3K8/Actin (left panel) and P-MEK/MEK (right panel) ratios in tumors from high- or low-MAP3K8 PDX, as assessed by densitometry analysis of the western blot shown in (a). P-values are based on Welch's t-test. Data are shown as means±s.e.m. (N=5 tumors per group). (c) Correlation between P-MEK/MEK and MAP3K8/Actin protein levels. Correlation coefficient σ and p-value are based on Spearman's rank correlation test. (d,e) Relative tumor volume over time of PDX models exhibiting either high- (d) or low-MAP3K8 (e) protein levels. Mice were either untreated (Ctrl) or subjected to treatment with MEK inhibitors, including AZD6244 and MEK162. P-values are based on Welch's t-test. * stands for p-value≦0.05 and **, p-value≦0.005. Data are shown as means±s.e.m (N≧8 mice per group). (f) Scatter plot of tumor growth inhibition (TGI) in AZD6244- and MEK162-treated groups, as compared to control group in high- or low-MAP3K8 PDX models, as indicated. P-values are based on Welch's t-test. Data are shown as means±s.e.m (N≧8 tumors per group). (g) The bar plot shows the number of mice without (grey) or with (black) lung metastases, as assessed by the detection of human-specific Alu sequences in RNA extracted from mouse lung samples. Mice from high- or low-MAP3K8 PDX models were either untreated (Ctrl) or treated with MEK inhibitors, AZD6244 or MEK162, as indicated (N≧8 mice per group of treatment). P-value is based on Fischer's exact test.

FIG. 6: Characteristics of subjects and tumor samples associated with low- or high-MAP3K8 protein levels: Association of low- or high-MAP3K8 human EOC with clinical data and remission status of patients from the Curie Institute cohort. The biological response was evaluated by CA-125 after the first round of treatment. Data are means of log values±s.e.m (N=24 tumors low-MAP3K8 and N=48 tumors high-MAP3K8). The clinical response was evaluated by the evolution of the tumor mass, determined by monitoring patients through their chemotherapeutic treatment; treatment was considered incomplete in patients with no or partial response to treatment. Debulking status was defined as optimal for tumor residues≦1 cm in diameter after resection, or as suboptimal for tumor residues >1 cm in diameter. P-values are based on Student's t-test for biological response and Fischer's exact test for all the other clinical features. ns, stand for “not significant”.

FIG. 7: MAP3K8 expression level in human EOCs: (a) Representative western blot showing MAP3K8 protein levels in human high-grade advanced stage EOC. Protein extraction was performed from tissues containing at least 73% of epithelial cancer cells, in average. Each human EOC has been annotated regarding the S&F signature (S=Stress and F=Fibrosis) or the DIMP signature (D=Differentiated; I=Immunoreactive; M=Mesenchymal and P=Proliferative), as indicated. Actin is used as an internal control for protein loading. (b) Representative views of MAP3K8 immunostaining from two distinct subgroups of “Stress” human ovarian tumors, according to MAP3K8 protein level, namely Stress-Low MAP3K8 (left panel) or Stress-High MAP3K8 (middle panel) as well as in Fibrosis-High MAP3K8 subgroup of EOC (right panel). Scale bars: 50 μm.

FIG. 8: BRAF amplification is not a readout for MEK activation: Scatter plot showing the level of P-MEK in human EOC without amplification of the BRAF gene (no BRAF amp, N=182 tumors) or exhibiting BRAF gene amplification (BRAF amp, N=25). P-value is based on Welch's t-test. Data are shown as means±s.e.m. (N=217 EOC). Data are from the TCGA (TCGA, 2011).

FIG. 9: MEK inhibitor treatment impairs tumor growth in high-MAP3K8 PDX models: (a) Western blot showing MAP3K8 protein levels in different tumors derived from 3 patient-derived xenograft (PDX) mouse models characterized as high-MAP3K8 protein levels, as indicated (N≧4 tumors per group), relative to the low-MAP3K8 PDX model previously shown (FIG. 5a). GAPDH is used as an internal control for protein loading. (b) Scatter plots of MAP3K8/GAPDH ratios in tumors from high- or low-MAP3K8 PDX, as assessed by densitometry analysis of the western blot shown in (a). P-values are based on Welch's t-test. Data are shown as means±s.e.m. (N≧5 tumors per group). (c-e) Relative tumor volume over time of high-MAP3K8 PDX1 (c), PDX2 (d) and PDX3 (e) models. Mice were either untreated (Ctrl) or treated with MEK inhibitors, such as AZD6244 and MEK162 as indicated. P-values are based on Welch's t-test. * stands for p-value≦0.05 and **, p-value≦0.005, *** stands for p-value≦0.0005. Data are shown as means±s.e.m (N≧4 mice per group).

FIG. 10: Main patient characteristics and clinicopathological features of EOCs in Curie cohort: Tumor samples were obtained from a cohort of consecutive ovarian carcinoma patients, treated at the Curie Institute between 1989 and 2005. For each patient, before chemotherapy, a surgical specimen was taken for pathological analysis and tumor tissue cryopreservation. The median's patient's age was 57 years (with a range of 31-80 years). Ovarian carcinomas were classified according to the World Health Organization histological classification of gynecological tumors. Pathological analysis identified 62 serous tumors (86.1%), 7 endometrioid tumors, 1 mucinous tumor, 1 malignant Brenner tumor and 1 borderline tumor. 68 subjects (94%) were classified has having a high histological grade (grade 2 and 3), and 4 subjects were classified has having a low histological grade (grade 1). 17 subjects (24%) were considered as early stage (International Federation of Gynaecology and Obstetrics (FIGO) I-IIc) and 55 subjects (76%) were considered as advanced stage (III and IV) of disease. Considering the S&F signature, 40 subjects were stratified as belonging to the “Fibrosis” subgroup of patients and 32 subjects as corresponding to the “oxidative stress response” subgroup of patients. Considering the DIMP signature, 15 patients were stratified as having tumor with the “Differentiated” signature, 22 patients as “Immunoreactive”, 23 patients as “Mesenchymal” and 12 patients as “Proliferative”. Patients were treated with combination of surgery and chemotherapy, the latter including alkylating or alkylating-like agents±taxane as first line treatment in most cases. All the subjects underwent surgery, 50 of them have a partial or suboptimal debulking and 22 subjects have a full or optimal debulking. 53 patients (73.6%) relapsed with a median delay of relapse of 21 months (with a range of 0.1-243 months).

FIG. 11: Quantification of MAP3K8 protein in low-MAP3K8 and high-MAP3K8:Quantification of MAP3K8 protein level as assessed by immunohistochemistry analysis in low-MAP3K8 and high-MAP3K8 subgroups of ovarian cancer patients. Histological scores (Hscores) the inventors are provided as a function of the percentage of positive cells multiplied by the staining intensity (ranging from 0 to 4). Two different investigators blindly evaluated three sections from distinct areas of each tumor.

### BRIEF DESCRIPTION OF THE INVENTION

Using transcriptomic and proteomic data analysis on a large set of EOC, the inventors revealed that MAP3K8 is a new prognostic marker. Indeed, they showed that both patient progression free survival (PFS) and overall survival (OS) are markedly decreased in patients with ovarian tumors exhibiting high MAP3K8 protein levels.

The inventors then investigated MAP3K8 function in tumorigenesis performing experiments in ovarian cancer cell lines using two complementary strategies: specific ATP-competitive kinase inhibition (KI) of MAP3K8; and stable knockdown of MAP3K8 expression via shRNA. They showed that MAP3K8 has a cell-autonomous function and controls proliferation, migration and invasion of EOC cells. In addition, tumor growth was severely reduced in mouse xenograft models upon MAP3K8 depletion through silencing, thereby demonstrating MAP3K8 pro-tumorigenic activity and ability to control tumor growth in vivo.

The inventors also explored MAP3K8 downstream signaling-pathways and determined that the main pathway involved in mediating MAP3K8 cell-autonomous function in ovarian cancer cell lines is the MEK/ERK pathway.

The inventors then verified that MAP3K8 protein expression level is associated with its kinase activity and with downstream signaling-pathways regulation. They effectively showed that MAP3K8 protein accumulation correlates with an increased kinase activity, as assessed by MAP3K8 phosphorylation state on 2 phosphorylation-sites required for MAP3K8 kinase activity. Importantly, they showed that MAP3K8 protein level correlates with MEK/ERK activation in human epithelial ovarian carcinomas. As MEK is a direct MAP3K8 substrate, these results suggest that the use of MEK inhibitors could be beneficial for EOC patients with high MAP3K8 expression levels.

In that respect, using mouse xenograft models, the inventors demonstrated that MAP3K8 cell-autonomous functions are pro-tumorigenic and that MAP3K8-dependent tumor growth is mediated by MEK activation. They, then, tested 2 different MEK inhibitors and showed, using patient derived EOC xenograft mouse models (PDX), that treatment with MEK inhibitors markedly reduces tumor growth in high MAP3K8 EOC, while it has no effect on low MAP3K8 EOC.

Therefore, the inventors showed that MAP3K8 can be used as a marker (i) for predicting clinical outcome of a patient affected with an ovarian cancer (ii) for selecting a subject affected with an ovarian cancer for a treatment comprising at least one MEK inhibitor or determining whether a subject affected with an ovarian cancer is susceptible to benefit from a treatment comprising at least one MEK inhibitor and/or (iii) for monitoring the response of a subject affected with an ovarian cancer to a treatment comprising at least one MEK inhibitor.

### Definitions

The methods of the invention as disclosed herein, may be in vivo, ex vivo or in vitro methods. Preferably, the methods of the invention are in vitro methods.

The term “cancer” or “tumor”, as used herein, refers to the presence of cells possessing characteristics typical of cancer-causing cells, such as uncontrolled proliferation, immortality, metastatic potential, rapid growth and proliferation rate, and some specific morphological features. The term “ovarian cancer” refers to any type of ovarian cancers, such as epithelial ovarian cancers (EOC), germ cell ovarian cancers, sex cord stromal ovarian cancers, fallopian tube or peritoneal cancers, and cancers derived from other organs, which spread to the ovaries (metastatic cancers).

An ovarian tumor is generally evaluated regarding its grade and stage. Knowing the stage and/or the grade of the tumor helps the doctors to decide on the best treatment of such tumor, and also gives a rough indication of the outcome.

The grade of an ovarian cancer gives an indication on how quickly it may develop. A sample of the ovarian tumor biopsy is typically analyzed under the microscope and the ovarian cancer may be graded as: Grade 1 (low-grade)—the ovarian cancer cells are growing slowly, look quite similar to normal cells (are well differentiated) and are less likely to spread than high-grade tumors; Grade 2 (moderate grade)—the ovarian cancer cells look more abnormal and are growing slightly more quickly; Grade 3 (high-grade)—the ovarian cancer cells are growing more quickly, look very abnormal (are poorly differentiated) and are more likely to spread than low-grade cancers.

The stage of an ovarian cancer is a term used to describe its size and whether it has spread beyond its original area in the body. There are typically four stages, Stages 1-4, for such tumor type. For stages 1-3, there are also sub-stages, which further describe the size and extent of the cancer. Stage 1 ovarian cancer only affects the ovaries. This stage is divided into three sub-groups: Stage 1a—the cancer is only in one ovary; Stage 1b—the cancer is in both ovaries; Stage 1c—the cancer is at either stage 1a or 1b, and there are cancer cells on the surface of one or both ovaries, or in the fluid taken from within the abdomen during surgery, or the ovary has burst (ruptured) before or during surgery. Stage 2 ovarian cancers have begun to spread outside the ovaries to other areas within the pelvis. There are three sub-groups: Stage 2a—the cancer has spread to the womb or fallopian tubes; Stage 2b—the cancer has spread to other structures within the pelvis, such as the rectum or bladder; Stage 2c—the cancer is at either stage 2a or 2b, and there are cancer cells in the fluid taken from within the abdomen during surgery. Stage 3 ovarian cancers have spread beyond the pelvis to the lining of the abdomen (a fatty membrane called the omentum), and/or to abdominal organs such as the lymph nodes in the abdomen, or the upper part of the bowel. There are three sub-groups: Stage 3a—the tumors in the abdomen are very small and cannot be seen except under a microscope; Stage 3b—the tumors in the abdomen can be seen but they are 2 cm or smaller; Stage 3c—the tumors in the abdomen are larger than 2 cm or they may have spread to nearby lymph nodes. Stage 4 the cancer has spread to other parts of the body such as the liver, lungs or distant lymph nodes (for example in the neck).

Preferably, the ovarian cancer in the context of the present invention is an EOC, deriving from the cells on the surface of the ovary. EOC is the most common form of ovarian cancer. EOC have been mainly classified regarding histological subtype, grade and stage. 70% of EOC are of serous histological subtype, more than 80% of which being high-grade (Malpica A., Am J Surg Pathol 2004; Hsu C. Y, Clin Cancer Res 2004; Malpica A., Am J Surg Pathol 2007).

Different subgroups of EOC have been identified by transcriptomic profiling. These subgroups are characterized by distinct molecular patterns, namely “S&F” (Stress and Fibrosis), “C1-C5”, or “DIMP” (Differentiated—Immunoreactive—Mesemchymal—Proliferative) signatures, reflecting mesenchymal features, epithelial differentiation, immune infiltrates or metabolic alterations (Mateescu B. et al, Nat Med 2011; TCGA, Nature 2011; Tothill et al, Clin Cancer Res 2008; Batista et al., Int J Biochem Cell Biol 2013; Verhaak et al, J Clin Invest 2013). “C1”, “Mesenchymal” and “Fibrosis” tumors are characterized by a desmoplastic reaction. Similarly, the “angiogenic” signatures identified in recent studies are enriched in extracellular matrix proteins (such as collagens and fibronectin) and associated with poor prognosis (Batista et al., Int J Biochem Cell Biol 2013; Bentink et al., PLoS One 7, e30269). Up to now, the only molecular mechanism deciphered is miR-200-dependent and is associated with the “S&F” subgroups of EOC patients (Batista et al., Int J Biochem Cell Biol 2013; Mateescu et al., Nat Med 2011).

As used herein, the term “MAP3K8” (Mitogen-activated protein kinase kinase kinase 8) also known as TPL2, TPL-2, COT, EST, ESTF, MEKK8 and c-COT refers to an enzyme that is encoded by the MAP3K8 gene (Gene ID: 1326) in humans. This gene is a proto-oncogene that encodes a member of the serine/threonine protein kinase family. The encoded protein localizes to the cytoplasm and can activate several signaling pathways including MAPK (Mitogen Activated Protein Kinase) and SAPK (Stress Activated Protein Kinase) pathways as it directly phosphorylates MEK-1, MEK-5, the JNK activator MKK-4 and the p38MAPK activator MKK-6 (Salmeron, 1996; Chiariello, 2000; Beinke, 2003; Waterfield, 2003; Jia, 2005) and further activates the downstream transcription factors NFAT (Nuclear Factor of Activated T cells) and NF-kappaB (Nuclear Factor Kappa-B) (Tsatsanis, 1998). This protein was also found to promote the production of TNF-alpha and IL-2 during T lymphocyte activation. Importantly, MAP3K8 controls downstream signaling-pathways in a cell-type- and stimulus-specific manner (Das, 2005; Kaiser, 2009). Indeed, in macrophages, MAP3K8 is specifically required for MEK/ERK activation (Dumitru, 2000), while in T-lymphocytes, MAP3K8 activates both MAPK and JNK signaling-pathways. This gene may also utilize a downstream in-frame translation start codon, and thus produce an iso form containing a shorter N-terminus. The shorter iso form has been shown to display weaker transforming activity. Alternate splicing results in multiple transcript variants that encode the same protein. Accession number corresponding to the human MAP3K8 transcript in Genbank is NM_005204.3, and accession number corresponding to the human MAP3K8 protein is NP_001231063.

As used herein, the term “MEK” (Mitogen-activated protein kinase kinase or MAP2K) refers to a dual-specificity kinase of the MAPK (Mitogen-activated protein kinase) pathway which regulates proliferation, gene expression, differentiation, mitosis, cell survival, and apoptosis. MEK is a protein kinase, which phosphorylates mitogen-activated protein kinase ERK (Extracellular-signal regulated kinase). MEK is encoded by seven genes: MAP2K1 (aka MEK1), MAP2K2 (aka MEK2), MAP2K3 (aka MKK3), MAP2K4 (aka MKK4), MAP2K5 (aka MKK5), MAP2K6 (aka MKK6) and MAP2K7 (aka MKK7). The activators of p38 (MKK3 and MKK6), JNK (MKK4 and MKK7), and ERK (MEK1 and MEK2) define independent MAP kinase signal transduction pathways. MEK is a direct substrate for MAP3 kinases such as RAF or MAP3K8 (Uniprot P41279).

As used herein, the term “ERK” (Extracellular-signal-regulated kinases) refers to widely expressed protein kinase intracellular signaling molecules that are involved in functions including the regulation of meiosis, mitosis, and post-mitotic functions in differentiated cells. MAPK1/ERK2 (Mitogen-activated protein kinase 1—P28482) and MAPK3/ERK1 (Mitogen-activated protein kinase 3—P27361) are the two MAPKs (85% sequence identity), which play an important role in the MEK/ERK cascade. They participate also in a signaling cascade initiated by activated KIT and KITLG/SCF. Depending on the cellular context, the MEK/ERK cascade mediates diverse biological functions such as cell growth, adhesion, survival and differentiation through the regulation of transcription, translation, and cytoskeletal rearrangements. The MEK/ERK cascade plays also a role in initiation and regulation of meiosis, mitosis, and post-mitotic functions in differentiated cells by phosphorylating a number of transcription factors.

About 160 substrates have already been discovered for ERK. Many of these substrates are localized in the nucleus, and seem to participate in the regulation of transcription upon stimulation. However, other substrates are found in the cytosol as well as in other cellular organelles, and those are responsible for processes such as translation, mitosis and apoptosis. Moreover, the MEK/ERK cascade is also involved in the regulation of the endosomal dynamics, including lysosome processing and endosome cycling through the perinuclear recycling compartment (PNRC), as well as in the fragmentation of the Golgi apparatus during mitosis. The substrates include transcription factors (such as ATF2, BCL6, ELK1, ERF, FOS, HSF4 or SPZ1), cytoskeletal elements (such as CANX, CTTN, GJA1, MAP2, MAPT, PXN, SORBS3 or STMN1), regulators of apoptosis (such as BAD, BTG2, CASP9, DAPK1, IER3, MCL1 or PPARG), regulators of translation (such as EIF4EBP1) and a variety of other signalling-related molecules (like ARHGEF2, DCC, FRS2 or GRB10). Protein kinases (such as RAFT, RPS6KA1/RSK1, RPS6KA3/RSK2, RPS6KA2/RSK3, RPS6KA6/RSK4, SYK, MKNK1/MNK1, MKNK2/MNK2, RPS6KA5/MSK1, RPS6KA4/MSK2, MAPKAPK3 or MAPKAPK5) and phosphatases (such as DUSP1, DUSP4, DUSP6 or DUSP16) are other substrates which enable the propagation the MEK/ERK signal to additional cytosolic and nuclear targets, thereby extending the specificity of the cascade. ERK mediates phosphorylation of TPR in response to EGF stimulation that may play a role in the spindle assembly checkpoint. ERK phosphorylates PML and promotes its interaction with PIN1, leading to PML degradation.

As used herein, the term “subject” or “patient” refers to an animal, preferably to a mammal, more preferably to a human, including adult, child and human at the prenatal stage, even more preferably to a female. However, the term “subject” can also refer to non-human animals, in particular mammals such as dogs, cats, horses, cows, pigs, sheeps and non-human primates, among others, that are in need of treatment.

The term “sample”, as used herein, means any sample containing cells derived from a subject, preferably a sample that contains nucleic acids. Examples of such samples include biopsies, organs, tissues, cell samples or cancer associated ascite fluids of an ovarian cancer. The sample may be treated prior to its use.

The term “cancer sample” refers to any sample comprising ovarian tumor cells derived from a patient, preferably an ovarian cancer sample that comprises protein. Preferably, the cancer sample contains only ovarian tumor cells (i.e., no normal or healthy cell).

As used herein, the term “progression free survival” refers to the time interval between that date of diagnosis and the first confirmed sign of disease recurrence.

As used herein, the term “overall survival” refers to the chances of staying alive for a group of individuals suffering from a cancer. It denotes the percentage of individuals in the group who are likely to be alive after a particular duration of time.

As used herein, the term “disease free survival” refers to the chances of staying free of disease after a particular treatment for a group of individuals suffering from a cancer. It is the percentage of individuals in the group who are likely to be free of disease after a specified duration of time. Disease-free survival rates are an indication of how effective a particular treatment is.

As used herein, the term “patient survival” refers to the time interval between the date of diagnosis and the date of death.

As used herein, the term “treatment”, “treat” or “treating” refers to any act intended to ameliorate the health status of patients suffering from an ovarian cancer such as surgery, therapy, prevention, prophylaxis and retardation of the ovarian cancer. In certain embodiments, such term refers to the amelioration or eradication of an ovarian cancer or symptoms associated with an ovarian cancer. In other embodiments, this term refers to minimizing the spread or worsening of the ovarian cancer resulting from the administration of one or more therapeutic agents to a subject with such an ovarian cancer.

As used herein, the term “surgery” refers to the main action to reduce the residual ovarian cancer to the lowest possible level. The term “surgical debulking”, as used herein, refers to as surgical removal of part of a malignant tumor which cannot be completely excised, in order to make subsequent therapy with drugs, radiation or other adjunctive measures more effective. The term “optimal debulking” refers to a surgery resulting in tumor residue of less than 1 cm in diameter after resection. “The term partial debulking” refers to a surgery resulting in tumor residue of more than 1 cm in diameter after resection.

The term “therapy”, as used herein, refers to any type of treatment of ovarian cancer (i.e., antitumoral therapy), including an adjuvant therapy and/or a neoadjuvant therapy. Therapy comprises radiotherapy and therapies, preferably systemic therapies such as hormone therapy, chemotherapy, immunotherapy and monoclonal antibody therapy.

The term “adjuvant therapy”, as used herein, refers to any type of treatment of ovarian cancer given as an additional treatment, usually after surgical resection of the primary tumor, in a patient affected with an ovarian cancer that is at risk of metastasizing and/or likely to recur. The aim of such an adjuvant treatment is to improve the prognosis. Adjuvant therapies comprise radiotherapy and therapy, preferably systemic therapy, such as hormone therapy, chemotherapy, immunotherapy and monoclonal antibody therapy.

The term “neoadjuvant therapy”, as used herein, refers to any type of treatment of ovarian cancer given prior to surgical resection of the primary tumor, in a patient affected with an ovarian cancer. The most common reason for neoadjuvant therapy is to reduce the size of the tumor so as to facilitate a more effective surgery. Neoadjuvant therapies comprise radiotherapy and therapy, preferably systemic therapy, such as hormone therapy, chemotherapy, immunotherapy and monoclonal antibody therapy.

As used herein, the term “chemotherapeutic treatment” or “chemotherapy” refers to a cancer therapeutic treatment using chemical or biochemical substances, in particular using one or several antineoplastic agents. Chemotherapy for ovarian cancer most often is a combination of 2 or more drugs, given for every 3- to 4-weeks. Giving 2 or more drugs in combination seems to be more effective in the initial treatment of ovarian cancer than giving just one drug alone. The standard approach is the combination of a platinum compound, such as cisplatin or carboplatin, and a taxane, such as paclitaxel (Taxol®) or docetaxel (Taxotere®). For intravenous (IV) chemotherapy, most doctors favor carboplatin over cisplatin because it has fewer side effects and is just as effective. Some of the chemo drugs that are helpful in treating ovarian cancer can be selected in the group comprising Albumin bound paclitaxel (nab-paclitaxel, Abraxane®), Altretamine (Hexalen®), Capecitabine (Xeloda®), Cyclophosphamide (Cytoxan®), Etoposide (VP-16), Gemcitabine (Gemzar®), Ifosfamide (Ifex®), Irinotecan (CPT-11, Camptosar®), Liposomal doxorubicin (Doxil®), Melphalan, Pemetrexed (Alimta®), Topotecan, and Vinorelbine (Navelbine®).

The term “radiotherapeutic treatment” or “radiotherapy” is a term commonly used in the art to refer to multiple types of radiation therapy including internal and external radiation therapies or radioimmunotherapy, and the use of various types of radiations including X-rays, gamma rays, alpha particles, beta particles, photons, electrons, neutrons, radioisotopes, and other forms of ionizing radiations.

The term “immunotherapy” refers to a cancer therapeutic treatment using the immune system to reject cancer. The therapeutic treatment stimulates the patient's immune system to attack the malignant tumor cells. It includes immunization of the patient with tumoral antigens (eg. by administering a cancer vaccine), in which case the patient's own immune system is trained to recognize tumor cells as targets to be destroyed, or administration of molecules stimulating the immune system such as cytokines, or administration of therapeutic antibodies as drugs, in which case the patient's immune system is recruited to destroy tumor cells by the therapeutic antibodies. In particular, antibodies are directed against specific antigens such as the unusual antigens that are presented on the surfaces of tumors. As illustrating example, one can cite Trastuzumab or Herceptin antibody, which is directed against HER2 and approved by FDA for treating breast cancer.

The term “monoclonal antibody therapy” refers to any antibody which function is to deplete tumor cells in a patient. In particular, therapeutic antibodies specifically bind to antigens present on the surface of the tumor cells, e.g. tumor specific antigens present predominantly or exclusively on tumor cells. Alternatively, therapeutic antibodies may also prevent tumor growth by blocking specific cell receptors.

The term “hormone therapy” or “hormonal therapy” refers to a cancer treatment having for purpose to block, add or remove hormones. For instance, in the case of ovarian cancer, especially ovarian stromal tumors, more rarely EOC, the hormone therapy can be Luteinizing-hormone-releasing hormone (LHRH) agonists such as goserelin (Zoladex®) and leuprolide (Lupron®); Tamoxifen or Aromatase inhibitors such as letrozole (Ferrara®), anastrozole (Arimidex®), and exemestane (Aromasin®).

By “effective amount” it is meant the necessary quantity of a compound or pharmaceutical composition according to the invention, which is able to achieve the desired effect, i.e. to prevent, remove, treat or reduce the deleterious effects of an ovarian cancer in mammals, including humans. It is understood that the administered dose may be adapted by those skilled in the art according to the patient, the pathology, the mode of administration, etc.

### DETAILED DESCRIPTION OF THE INVENTION

The present invention refers to ovarian cancer, especially to EOC and preferably to high-grade and/or advanced stage epithelial ovarian cancer.

Using proteomic data analysis, the inventors have demonstrated, in the experimental section below, that MAP3K8 expression levels predict disease outcome (patient progression free survival and overall survival) in human ovarian cancers better than standard prognostic markers.

The present invention therefore relates to the use of MAP3K8 as a prognostic marker in ovarian cancers, preferably in EOC, even more preferably in EOC of high-grade and/or advanced-stage.

As used herein, the term “prognostic marker” refers to an expressed molecule, i.e. MAP3K8, used to predict or monitor clinical outcome of a subject affected with an ovarian cancer.

The present invention also relates to a method for predicting the clinical outcome of a subject affected with an ovarian cancer, wherein the method comprises the step of determining the expression level and/or the activity level of MAP3K8 in a cancer sample from said subject, a high expression level and/or a high activity level of MAP3K8 being indicative of poor prognosis.

As used herein, the term “poor prognosis” refers to a patient decreased progression free survival and/or a decreased overall survival and/or an early disease progression and/or an increased disease recurrence and/or an increased metastasis formation and/or an increased tumor growth in comparison to a population of patients suffering from the same cancer and having the same treatment. In the contrary, the term “good prognosis” refers to a patient increased progression free survival and/or an increased overall survival and/or a decreased tumor growth and/or a decreased tumor progression and/or an decreased disease recurrence and/or an decreased metastasis formation in comparison to a population of patients suffering from the same cancer and having the same treatment.

In addition, the inventors have demonstrated (i.e. by western blot analysis of ratio P-MEK/total MEK, detailed in the experimental section below) that MAP3K8-dependent tumor growth is mediated by MEK activation and that tumors with high MAP3K8 expression levels present a constitutive activation of MEK signaling, suggesting MAP3K8 can be used as a marker for (i) selecting patients susceptible to benefit from a treatment comprising at least one MEK inhibitor and for (ii) monitoring the response of said treatment. In addition, tumors presenting high levels of MAP3K8 expression were responsive to treatment with 2 different MEK inhibitors.

As demonstrated in the experimental data below, the inventors have shown that MEK/ERK pathway is the main pathway activated downstream in mediating MAP3K8 pro-tumorigenic properties in ovarian cancer and that MEK, a direct MAP3K8 substrate, is constitutively active in ovarian cancer with high-MAP3K8 protein levels. They further demonstrated that the inhibition of MEK with two different ATP-non competitive MEK inhibitors (i.e. AZD6244/Selumetinib and MEK162; both tested in clinical trials for treatment of low-grade ovarian tumor patients (Farley et al, Lancet Onco 2013)) markedly reduced tumor growth (i.e. 60% tumor growth inhibition) in high-MAP3K8 ovarian tumor (i.e. high-MAP3K8 PDX model). As ERK is a MEK direct substrate, the P-ERK/ERK ratio can be detected in order to monitoring the response of the subject to the treatment comprising at least one MEK inhibitor.

In one embodiment, the present invention concerns also the use of MAP3K8 as a marker for selecting a subject affected with an ovarian cancer for a treatment comprising at least one MEK inhibitor, or determining whether a subject affected with an ovarian cancer is susceptible to benefit from a treatment comprising at least one MEK inhibitor, preferably comprising the step of determining the expression level and/or the activity level of MAP3K8, and wherein a high expression level and/or a high activity level of MAP3K8 indicates that said subject is susceptible to benefit from said treatment.

In a further aspect, the present invention concerns a method for selecting a subject affected with an ovarian cancer for a treatment comprising at least one MEK inhibitor, or determining whether a subject affected with an ovarian cancer is susceptible to benefit from a treatment comprising at least one MEK inhibitor, wherein the method comprises the step of determining the expression level and/or the activity level of MAP3K8 in a cancer sample from said subject, a high expression level and/or a high activity level of MAP3K8 being indicative that said subject is susceptible to benefit from said treatment.

As used herein, the term “expression level of MAP3K8” refers to the expression level of MAP3K8 protein. This term also refers to the expression level of phospho-MAP3K8 protein (P-MAP3K8). In the context of the invention, a high expression level of MAP3K8 is indicative of a poor prognosis for ovarian cancer patients (i.e. decreased progression free survival and/or a decreased overall survival and/or an early disease progression and/or an increased disease recurrence and/or an increased metastasis formation and/or an increased tumor growth). The expression level of MAP3K8 can be determined from a sample, including an ovarian cancer sample by a variety of techniques well known by the man skilled in the art.

In an embodiment, the expression level of MAP3K8 is determined by measuring the quantity of MAP3K8 protein. The quantity of protein may be measured by any methods known by the skilled person. Usually, these methods comprise contacting the ovarian cancer sample with a binding partner capable of selectively interacting with MAP3K8 protein present in said sample.

The binding partner is generally a polyclonal or monoclonal antibody, preferably a monoclonal antibody. Polyclonal and monoclonal antibodies against MAP3K8 are commercially available. Examples of marketed antibodies are Cot (M-20) ref Sc-720 from Santa Cruz or anti-MAP3K8 antibody ref ab49152 from Abcam or MAP3K8 oligoclonal antibody clone 3HCLC ref 710377 from lifetechnologies. In addition, the methods for producing anti-MAP3K8 antibodies are well known in the art. In a preferred embodiment, such antibody is specific to MAP3K8.

The quantity of MAP3K8 protein and/or P-MAP3K8 may be measured by semi-quantitative Western blots, immunochemistry (enzyme-labeled and mediated immunoassays, such as ELISAs, biotin/avidin type assays, radioimmunoassay, immunoelectrophoresis or immunoprecipitation) or by protein or antibody arrays. The protein expression level may be assessed by immunohistochemistry on a tissue section of the cancer sample (e.g. frozen or formalin-fixed paraffin embedded material). The reactions generally include revealing labels such as fluorescent, chemiluminescent, radioactive, enzymatic labels or dye molecules, or other methods for detecting the formation of a complex between the antigen and the antibody or antibodies reacted therewith. Preferably, the quantity of MAP3K8 protein is measured by immunohistochemistry or semi-quantitative western-blot. MAP3K8 detection by immunohistochemistry may require an antigen-unmasking step. More preferably, the quantity of MAP3K8 protein is measured by semi-quantitative Western blots.

As used herein, the term “activity level of MAP3K8” refers to the kinase activity of MAP3K8, particularly to the enzymatic capacity of MAP3K8 to catalyze the following reaction: ATP+a protein⇄ADP+a phosphoprotein. This reaction requires magnesium as a cofactor.

The activity level of MAP3K8 can be determined from a sample, including an ovarian cancer sample. The activity level of MAP3K8 may be measured by any methods known by the skilled person. For example, the activity level of MAP3K8 can be measured by enzyme assays such as spectrophotometry, fluorometry, calorimetry, chemiluminescence, static light scattering, microscale thermophoresis, radiometry or chromatography. In one embodiment, the activity of MAP3K8 is measured by analyzing the activation of the MEK/ERK pathway. Preferably, the activity level of MAP3K8 is determined by measuring the phosphorylated level of MEK or ERK (so-called P-MEK/MEK or P-ERK/ERK ratios). Methods for determining the phosphorylated level of proteins is well known in the art such as Western blot, ELISA (Enzyme-Linked Immunosorbent Assay), Cell-based ELISA, intracellular flow cytometry and mass spectrometry. Western blot is the preferred technique. For example, said P-MEK/MEK or P-ERK/ERK ratios can be determined using specific antibody. Particularly, P-MEK and ERK can be determined by using polyclonal antibodies; MEK and P-ERK can be determined by using monoclonal antibodies.

In an embodiment of these above-mentioned methods, the methods may further comprise the step of comparing the expression level and/or the activity level of MAP3K8 protein to a reference expression level and/or a reference activity level.

The methods of the invention may also further comprise determining whether the expression level and/or the activity level of MAP3K8, is high or low compared to the reference expression and/or activity level respectively.

In such embodiment, the expression and/or activity level of MAP3K8 in the patient sample of interest is considered as high if the level or quantity of MAP3K8 is above a cut-off value easily adjusted by the skilled person depending on the reference level of interest. The cut-off value may be defined, for example, according to the average and the variance of the expression rates of MAP3K8 in each population (“high MAP3K8” pattern versus “low Map3K8” pattern).

In an embodiment, the reference expression level and/or the reference activity level is the expression and/or activity level respectively of MAP3K8 in a control sample.

The control sample can be a non-tumoral sample, preferably from the same tissue type than the cancer sample (i.e. ovarian tissue). The control sample may be obtained from the subject affected with the ovarian cancer (i.e. sample from the other non-tumoral ovary if available) or from another subject, preferably a normal or healthy subject, i.e. a subject who does not suffer from an ovarian cancer. Preferably, the control sample is obtained from the same subject than the ovarian cancer sample.

The control sample can also be a tumoral sample, preferably from the same tissue type than the cancer sample (i.e. ovarian tissue). In the case of the present invention, the control sample is preferably obtained from a subject affected with ovarian cancer.

In a further embodiment, the reference expression and/or activity level is determined from the expression level of MAP3K8 among a population of randomly selected cancer samples or among a validated reference population, i.e. a population which belongs to the “low MAP3K8” or to the “high MAP3K8” subgroup of EOC patients, using statistical analysis well known from the person skilled in the art

For example when MAP3K8 protein level is obtained by western blot analysis, statistical analysis such as Kaplan-Meier analyses can be performed using successive iterations to find the optimal sample size thresholds that maximally discriminate the “low” and the “high” subgroups of patients. The reference expression and/or activity value, i.e. value above which a patient's MAP3K8 level is considered high or below which a patient's MAP3K8 level is considered low according to the methods of the invention, is thus defined as this one that maximally discriminates the two patient subsets.

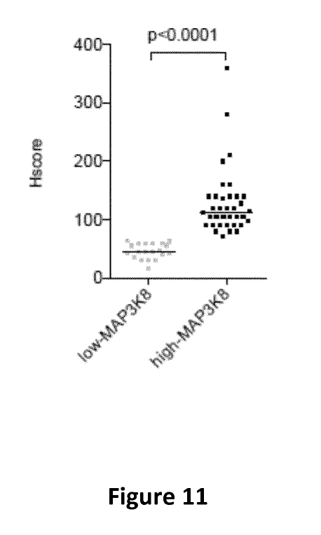

In another example when MAP3K8 protein level is obtained by immunohistochemistry analysis, Hscore is defined as the intensity of the staining, i.e. MAP3K8 protein expression intensity (comprised ranging from 0 to 4), multiplied by the percentage of cells stained, i.e. expressing MAP3K8 protein. The reference expression and/or activity value is defined as an Hscore of 90 (see FIG. 11).

Using these above-mentioned methods, the inventors have identified in a population of randomly selected cancer samples that the low-MAP3K8 group and high-MAP3K8 group respectively represent 33% and 67% of patients respectively.

As used herein, the term “population of randomly selected cancer samples” refers to a population comprising at least 50 samples, more preferably at least 100, 200 or 250 samples. In a preferred embodiment, the population of randomly selected cancer samples contains only one type of tumors (i.e. tumors derived from the cells of a same organ), preferably of the same type than the tumor of the patient. In a particular embodiment, the patient is affected with an ovarian cancer and the population of randomly selected cancer samples comprises only ovarian cancer samples.

Optionally, before comparison with the reference expression and/or activity level, the expression levels of proteins, in particular of MAP3K8 protein, are normalized using the expression level of an endogenous control protein having a stable expression in different cancer samples, such as GAPDH (glyceraldehyde 3-phosphate dehydrogenase) and Actin. This optionally step is preferably used in the case of Western blot analysis.

The present invention relates to a method for monitoring the response of a subject affected with an ovarian cancer with a high expression level and/or activity level of MAP3K8 (i.e. an ovarian cancer with a high expression level and/or activity level of MAP3K8), to a treatment comprising at least one MEK inhibitor, wherein the method comprises the step of determining the P-ERK/ERK ratio, in an ovarian cancer sample from said subject, a low P-ERK/ERK ratio being indicative that the subject is responsive to the treatment comprising at least one MEK inhibitor.

The present invention also relates to a method for monitoring the response of a subject affected with an ovarian cancer with a high expression level and/or a high activity level of MAP3K8 to a treatment comprising at least one MEK inhibitor, wherein the method comprises the step of determining P-ERK/ERK ratio, in a cancer sample from said subject, a high P-ERK/ERK ratio being indicative that the subject is not responsive and/or resistant to said treatment comprising at least one MEK inhibitor.

The monitoring of the response to the treatment comprising at least one MEK inhibitor can be determined as follow. A first ovarian cancer sample is obtained from the subject before the administration of the treatment comprising at least one MEK inhibitor. A second ovarian cancer sample is obtained from the same subject after the administration of the treatment comprising at least one MEK inhibitor. Preferably, the second ovarian cancer sample is collected when a significant effect on tumor growth can be expected with said treatment. The timing for collecting the second ovarian cancer sample is determined by the man of the art. For example, the second ovarian cancer sample can be collected 2 or 4 weeks after the administration of the treatment comprising at least one MEK inhibitor. The responsiveness of the patient to said treatment is evaluated by technics well known from the man from the art. For example, the P-ERK quantity can be determined by immunohistochemistry (IHC) by using a P-ERK monoclonal antibody such as P-ERK antibody reference 9106 from Cell signaling, or the P-ERK/ERK ratio can be determined by densitometry analysis of Western Blot. The level of P-ERK or P-ERK/ERK ratio obtained will be determined; using the above-mentioned methods, in the sample collected prior treatment and in the samples collected after treatment. The low or high level of P-ERK or P-ERK/ERK ratio will be determined in the samples collected after treatment relative to the sample, from the same patient, collected prior treatment.

A lower expression level of P-ERK in tumor cells contained in the sample collected after the said treatment in comparison with the expression level of P-ERK in tumor cells contained in the sample collected before the said treatment indicates that the subject is responsive to the treatment comprising at least one MEK inhibitor. On the contrary, a high expression level of P-ERK in tumor cells contained in the sample collected after the said treatment in comparison with the expression level of P-ERK in tumor cells contained in the sample collected before the said treatment indicates that the subject is not responsive to the treatment comprising at least one MEK inhibitor and/or that the subject is resistant to the treatment.

This method may also provide an indication of the required duration and/or intensity of the treatment comprising at least one MEK inhibitor. In this case, a follow-up after each cycle of said treatment with the determination of the P-ERK/ERK ratio compared to the same parameter before said cycle of said treatment will help to adjust the treatment duration and/or intensity/dosage accordingly.

In particular, this method could be indicative of the efficacy of the treatment comprising at least one MEK inhibitor for increasing the progression free survival and/or disease free interval and/or, the overall survival, and/or for decreasing the disease or metastasis occurrence and/or tumor growth and/or tumor progression.

In addition, the invention also relates to kits, and their uses, in particular to practice all the above-mentioned methods of the invention.

In an embodiment, the invention relates to a kit and its use (a) for predicting clinical outcome of a subject affected with an ovarian cancer, and/or (b) for selecting a subject affected with an ovarian cancer for a treatment comprising at least one MEK inhibitor, or determining whether a subject affected with an ovarian cancer is susceptible to benefit from a treatment comprising at least one MEK inhibitor, wherein the kit comprises means for detecting the expression level and/or activity level of MAP3K8, preferably means specific for detecting MAP3K8's expression and/or activity, more preferably comprising at least one antibody specific to MAP3K8 protein and optionally, a leaflet providing guidelines to use such a kit.

In another embodiment, the invention relates to a kit and its use for monitoring the response to a treatment comprising at least one MEK inhibitor of a subject affected with an ovarian cancer, particularly an ovarian cancer with a high expression level and/or activity level of MAP3K8, wherein the kit comprises:

(i) at least one antibody specific to P-ERK or ERK; and/or

(ii) at least one probe specific to ERK mRNA or cDNA; and/or

(iii) at least one nucleic acid primer pair specific to ERK mRNA or cDNA; and optionally, a leaflet providing guidelines to use such a kit.

The kits of the invention may further comprise means for detecting the formation of complexes between proteins (i.e. MAP3K8 or ERK) and their specific antibodies (i.e. antibodies specific to MAP3K8 or P-MAP3K8 or ERK or P-ERK).

The kit for monitoring the response to a treatment comprising at least one MEK inhibitor may further comprise (i) means for detecting the hybridization of the probes with the ERK mRNA or cDNA; and/or (ii) means for amplifying and/or detecting the ERK mRNA or cDNA molecules by using their pairs of primers.

The kits of the invention can further comprise control reagents and other necessary reagents.

The kits of the invention may also comprise a computer readable medium comprising computer-executable instructions for performing the method of the invention for predicting clinical outcome of a subject affected with an ovarian cancer and/or for selecting a subject affected with an ovarian cancer for a treatment comprising at least one MEK inhibitor or determining whether a subject affected with an ovarian cancer is susceptible to benefit from a treatment comprising at least one MEK inhibitor, and/or for monitoring the response to a treatment comprising at least one MEK inhibitor of a subject affected with an ovarian cancer.

As used herein and below, the term “antibody” includes monoclonal antibodies, chimeric antibodies, humanized antibodies, recombinant antibodies and fragments thereof. Antibody fragment means, for example, F(ab)2, Fab, Fab′ or sFv fragments. According to a particular embodiment, the antibody can be IgG, IgM, IgA, IgD or IgE, preferably IgG or IgM. Methods for producing antibodies are well known to those persons skilled in the art.

Antibodies specific of MAP3K8, P-MAP3K8, ERK and P-ERK have been disclosed in details above. However, in order to be effective, such antibodies might have to be engineered to be able to penetrate the cellular membrane into the nucleus.

In an embodiment of these above-mentioned methods, the methods further comprise the step of providing an ovarian cancer sample from the subject.

In a further embodiment of these above mentioned methods, the methods further comprise the step of comparing the expression level to a reference expression level.

The inventors have also shown that inhibition of MEK expression in ovarian cancer, preferably in ovarian cancer with high expression level and/or high activity level of MAP3K8 significantly reduces tumor growth and lung metastatic incidence.

An aspect of the present invention then relates to a MEK inhibitor for use in the treatment of an ovarian cancer with a high expression level and/or a high activity level of MAP3K8.

A further aspect of the present invention relates to a MEK inhibitor for use in a treatment for reducing and/or preventing lung metastatic incidence related to ovarian cancer.

Accordingly, the MEK inhibitor can be, without being limiting thereto, a small molecule, an antibody, a nucleic acid, an aptamer, a peptide, a polypeptide, a protein or any molecule preventing the interaction of MEK with a MEK interacting partner (such as ERK or MAP3K8).

In one embodiment, said MEK inhibitor is selected from the group consisting of a small molecule, an antibody against MEK and a nucleic acid molecule interfering specifically with MEK expression such as an antisense against MEK, a siRNA against MEK and a shRNA against MEK. Preferably, said MEK inhibitor is a small molecule.

The term “small molecule” refers to a molecule of less than 1,000 daltons, in particular organic or inorganic compounds. Structural design in chemistry is helpful to find such a molecule.

Small molecules inhibiting MEK may be selected, without being limiting thereto, from the group consisting of artigenin, AS703026, AZD8330, AZD6244 (Selumetinib), BAY 869766, KT 5720, AS-252424, BIX 02189, Debromohymenialdisine, Hypothemycin, MEK Inhibiteur II, PD 0325901, PD 184,352, SB 203580, PD 184161, PD 198306, PD 98059, PD 318088 Selumetinib, SL-327, TAK-733, Trametinib, U-0126, U-0124, 2-Bromoaldisine, Myricetin, Chk2 Inhibiteur, Honokiol, cobimetinib, XL518, CI-1040 and MEK162. Preferentially, said small molecule is selected from the group consisting of PD 0325901, AZD6244 and MEK162.

In one embodiment of the present invention, the MEK inhibitor is a nucleic acid molecule interfering specifically with MEK expression or with the interaction between MEK and one of their specific partners. For example a MEK inhibitor can be a shRNA against MEK. Such nucleic acids are more amply detailed below. Preferably this nucleic acid is selected from the group consisting of RNAi, an antisense nucleic acid or a ribozyme. Said nucleic acid can have a sequence from 15 to 50 nucleotides, preferably from 15 to 30 nucleotides.

The term “RNAi” or “interfering RNA” means any RNA, which is capable of down-regulating the expression of the targeted protein. It encompasses small interfering RNA (siRNA), double-stranded RNA (dsRNA), single-stranded RNA (ssRNA), micro-RNA (miRNA), and short hairpin RNA (shRNA) molecules. RNA interference, designates a phenomenon by which dsRNA specifically suppresses expression of a target gene at post-translational level. In normal conditions, RNA interference is initiated by double-stranded RNA molecules (dsRNA) of several thousand base pairs in length. In vivo, dsRNA introduced into a cell is cleaved into a mixture of short dsRNA molecules called siRNA. The enzyme that catalyzes the cleavage, Dicer, is an endo-RNase that contains RNase III domains (Bernstein et al., Nature 2001). In mammalian cells, the siRNAs produced by Dicer are 21-23 base pair in length, with a 19 or 20 nucleotides duplex sequence, two-nucleotide 3′ overhangs and 5′-triphosphate extremities (Elbashir et al., EMBO J 2001; Elbashir et al., Nature 2001; Zamore et al., Cell 2000). A number of patents and patent applications have described, in general terms, the use of siRNA molecules to inhibit gene expression, for example, WO 99/32619, US 20040053876, US 20040102408 and WO 2004/007718.

siRNA are usually designed against a region 50-100 nucleotides downstream the translation initiator codon, whereas 5′UTR (untranslated region) and 3′UTR are usually avoided. The chosen siRNA target sequence should be subjected to a BLAST search against EST database to ensure that the only desired gene is targeted. Various products are commercially available to aid in the preparation and use of siRNA. In a preferred embodiment, the RNAi molecule is a siRNA of at least about 15-50 nucleotides in length, preferably about 20-30 base nucleotides.

RNAi can comprise naturally occurring RNA, synthetic RNA, or recombinantly produced RNA, as well as altered RNA that differs from naturally-occurring RNA by the addition, deletion, substitution and/or alteration of one or more nucleotides. Such alterations can include addition of non-nucleotide material, such as to the end of the molecule or to one or more internal nucleotides of the RNAi, including modifications that make the RNAi resistant to nuclease digestion.

RNAi may be administered in free (naked) form or by the use of delivery systems that enhance stability and/or targeting, e.g., liposomes, or incorporated into other vehicles, such as hydrogels, cyclodextrins, biodegradable nanocapsules, bioadhesive microspheres, or proteinaceous vectors (WO 00/53722), or in combination with a cationic peptide (US 2007275923). They may also be administered in the form of their precursors or encoding DNAs.

Antisense nucleic acid can also be used to down-regulate the expression of MEK. The antisense nucleic acid can be complementary to all or part of a sense nucleic acid encoding MEK e.g., complementary to the coding strand of a double-stranded cDNA molecule or complementary to an mRNA sequence, and is thought to interfere with the translation of the target mRNA. Preferably, the antisense nucleic acid is an RNA molecule complementary to a target mRNA encoding MEK.

An antisense nucleic acid can be, for example, about 5, 10, 15, 20, 25, 30, 35, 40, 45 or 50 nucleotides in length. Particularly, antisense RNA molecules are usually 18-50 nucleotides in length.

An antisense nucleic acid can be constructed using chemical synthesis and enzymatic ligation reactions using procedures known in the art. Particularly, antisense RNA can be chemically synthesized, produced by in vitro transcription from linear (e.g. PCR products) or circular templates (e.g., viral or non-viral vectors), or produced by in vivo transcription from viral or non-viral vectors.

Antisense nucleic acid may be modified to have enhanced stability, nuclease resistance, target specificity and improved pharmacological properties. For example, antisense nucleic acid may include modified nucleotides designed to increase the physical stability of the duplex formed between the antisense and sense nucleic acids, e.g., phosphorothioate derivatives and acridine substituted nucleotides.

Ribozyme molecules can also be used to block the expression of MEK. Ribozymes are catalytic RNA molecules with ribonuclease activity which are capable of cleaving a single-stranded nucleic acid, such as an mRNA, to which they have a complementary region. Thus, ribozymes can be used to catalytically cleave mRNA transcripts to thereby inhibit translation of the protein encoded by the mRNA. Ribozyme molecules specific for MEK can be designed, produced, and administered by methods commonly known to the art (see e.g., Fanning and Symonds, Springer 2006, reviewing therapeutic use of hammerhead ribozymes and small hairpin RNA).

In a particular embodiment, a vector, preferably a viral vector comprising a construct allowing the expression of interfering nucleic acid molecule, expresses the interfering nucleic acid molecule. For instance, the viral vector can be an adenovirus, an adeno-associated virus, a lentivirus or a herpes simplex virus.

The term “aptamer” refers to a molecule of nucleic acid or a peptide able to bind specifically to MEK proteins or to a binding partner of MEK. In a preferred embodiment, the aptamers are nucleic acids, preferably RNA, generally comprising between 5 and 120 nucleotides (Osborne et al., Curr Opin Chem Biol 1997). They can be selected in vitro according to a process known as SELEX (Systematic Evolution of Ligands by Exponential Enrichment).

In a further aspect of the present invention, the MEK inhibitor is used in combination with any other type of treatment suitable to treat ovarian cancer, including surgery, radiotherapy or tumor chemotherapy agent such as those mentioned above.

The invention also relates to i) a method for treating an ovarian cancer, preferably an ovarian cancer with a high level of MAP3K8, by administering a therapeutic effective amount of a MEK inhibitor and ii) to the use of a MEK inhibitor for treating an ovarian cancer, preferably an ovarian cancer with a high level of MAP3K8. In particular, the treatment allows the improvement of the clinical outcomes of a patient having an ovarian cancer, preferably an EOC, more preferably a high-grade and/or advanced stage EOC, more preferably an ovarian cancer with a high level of MAP3K8.

The present invention also relates to:
a pharmaceutical composition comprising at least one MEK inhibitor, and optionally a pharmaceutically carrier, for use in the treatment of an ovarian cancer, preferably an ovarian cancer with a high expression level and/or a high activity level of MAP3K8.
a MEK inhibitor, and optionally a pharmaceutically acceptable carrier, for use in the treatment of an ovarian cancer, preferably an ovarian cancer with a high expression level and/or a high activity level of MAP3K8, optionally in combination with radiotherapy or an anti-tumoral agent.
the use of a MEK inhibitor for the manufacture of a medicament for the treatment of an ovarian cancer, preferably an ovarian cancer with a high expression level and/or activity level of MAP3K8, optionally in combination with radiotherapy or an anti-tumoral agent.
a method for treating an ovarian cancer, preferably an ovarian cancer with a high expression level and/or a high activity level of MAP3K8, in a subject in need thereof, comprising administering an effective amount of a pharmaceutical composition comprising a MEK inhibitor and optionally a pharmaceutically acceptable carrier.
a combined preparation, product or kit containing (a) a MEK inhibitor and (b) an anti-tumoral agent as a combined preparation for simultaneous, separate or sequential use in the treatment of an ovarian cancer, preferably an ovarian cancer with a high expression level and/or a high activity level of MAP3K8.
a method for treating an ovarian cancer, preferably an ovarian cancer with a high expression level and/or a high activity level of MAP3K8, in a subject in need thereof, comprising administering an effective amount of a pharmaceutical composition comprising a MEK inhibitor, and an effective amount of a pharmaceutical composition comprising an anti-tumoral agent.
a method for treating an ovarian cancer, preferably an ovarian cancer with a high expression level and/or a high activity level of MAP3K8, in a subject in need thereof, comprising administering an effective amount of a pharmaceutical composition comprising a MEK inhibitor in combination with radiotherapy.

Optionally, these methods may further comprise the step of determining the expression levels and/or activity level of MAP3K8 in a cancer sample from said patient. The methods may further comprise the step of selecting a patient according the expression level and/or activity level of MAP3K8 in a cancer sample from said patient.

Preferably, the ovarian cancer according to these above-mentioned methods is an EOC, more preferably a high-grade and/or advanced-stage EOC.

By “pharmaceutically” is intended molecular entities and compositions that do not produce adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

By a “therapeutically efficient amount” is meant an amount of compound administered to a patient able to treat and/or to prevent, reduce and/or alleviate one or more of the symptoms of cancer and/or metastasis, preferably an ovarian cancer.

It will be understood that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed, the age, body weight, general health, sex and diet of the patient; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieve. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustments of the dosage to the patient to be treated. A medicament typically contains from about 0.01 mg to about 500 mg of the active ingredient, preferably from 1 mg to about 100 mg of the active ingredient. An effective amount of the drugs is ordinarily supplied at a dosage level from 0.0002 mg/Kg to about 20 mg/kg of body weight per day, especially from about 0.001 mg/kg to 7 mg/kg of body weight per day.

The form of the pharmaceutical compositions, the route of administration, the dosage and the regiment naturally depend upon the condition to be treated, the severity of the illness, the age, weight, and sex of the patient, etc.

The pharmaceutical compositions of the invention can be formulated for a topical, oral, intranasal, parenteral, intraocular, intravenous, intramuscular or subcutaneous administration and the like.

Further aspects and advantages of the present invention will be described in the following examples, which should be regarded as illustrative and not limiting.

### Example

Materials and Methods

Cohorts of Epithelial Ovarian Carcinoma Patients

Ovarian tumors were obtained from a cohort of patients treated at the Curie Institute between 1989 and 2005. Patient characteristics and clinical features have been previously described (Batista L. et al., Int J Biochem Cell Biol 2013; Mateescu B. et al., Nat Med 2011). The Institutional Review Board and Ethics committee approved analyses based on the Curie cohort. Before inclusion in the study, patients were informed that their biological samples could be used for research purposes and that they had the right to refuse if they so wished. Analysis of tumor samples was performed according to the relevant national law on the protection of people taking part in biomedical research. Patient characteristics and clinical features of the cohorts referred to as TCGA have been previously described (TCGA, Nature 2011; Tothill R. W. et al., Clin Cancer Res 2008; Verhaak R. G. et al., J Clin Invest 2013)

Immunohistochemical Stainings

Sections of paraffin-embedded tissue (3 μm) were stained using a streptavidin-peroxidase protocol and the Benchmark immunostainer (Ventana, Illkirch, France) with specific antibodies recognizing MAP3K8 (1/100, Santa Cruz #sc-720) following unmasking with EDTA. For staining quantification, at least five distinct areas of each tumour were evaluated by two different investigators. A score, named Hscore for “Histological score”, was given as a function of the percentage of positive tumor epithelial cells and the staining intensity from 0 to 4.

Production of Lentiviral Vector and Generation of Stable Cell Lines

PLKO.1-derived vectors with 2 different shRNAs targeting human MAP3K8 (TRCN0000010012 and TRCN0000010013 for shMAP3K8_1 and shMAP3K8_2 respectively), or expressing a scrambled shRNA (shCtrl), were purchased from Sigma-Aldrich. Viruses were produced by co-transfection (with Lipofectamine 2000, Invitrogen) of 293T cells with the vector plasmid, a vesicular stomatitis virus envelope expression plasmid (Vsvg), and a second-generation packaging plasmid (pPax2). Purified viral particles were used at MOI 5 to infect SKOV3 cells overnight. Infected cells were selected with puromycin (1 μg/mL) for one week, prior to experimental use.

Cell Culture, Treatments, Growth Kinetics and Cell Cycle Analysis

The human epithelial ovarian cancer cell line SKOV3 and the SKOV3 stable cell lines were propagated in DMEM (Gibco) supplemented with 10% fetal bovine serum (FBS, PAA), penicillin and streptomycin (Gibco). 1 μg/mL puromycin was added only for SKOV3 stable cell lines. For SKOV3 stable cell lines, 10% serum stimulation was performed after overnight serum starvation for the indicated times. SKOV3 cells were plated and immediately treated or not with 5 μM MAP3K8 kinase inhibitor (KI, Calbiochem #616373) for the indicated times. The number of living cells was measured by trypan blue exclusion using Vi-Cell analyzer (Beckman Coulter). Cell cycle distributions were performed on ethanol-fixed cells, stained with propidium iodide and analyzed by flow cytometry. Flow cytometry data were acquired using CellQuest Pro (Becton Dickinson) software on the FACS LSRII machine (Becton Dickinson) and were analyzed using ModfitLT (Verity) software.

Mouse Ovarian Surface Epithelial cell lines (MOSEC) were a kind gift from Dr. K. Roby and P. Terranova (University of Kansas) (Roby K. F. et al., Carcinogenesis 2000). Briefly, cells were obtained by gentle trypsinization of mouse ovaries. After repeated passages in vitro, cells undergo spontaneous transformation and become tumorigenic. MOSEC were propagated in Dulbecco's Modified Eagles Medium (DMEM Gibco #41966-029) supplemented with 4% of fetal bovine serum (FBS), penicillin (100 U/mL), streptomycin (100 ug/mL), insulin (5 μg/mL), transferrin (5 μg/mL) and sodium selenite (5 ng/mL) (ITS mix, Sigma Aldrich #I-1884).

Migration and Invasion Assays

12 wells cell culture insert, and 24 wells Transwell BioCoat™ growth factor reduced Matrigel™ invasion chambers (BD Biosciences, 8 μM Pore size) were used for migration and invasion assays, respectively. After overnight serum starvation, cells were plated to the upper side of the Transwell device, at least in duplicates, in serum free medium, while the lower well contained regular 10% FBS culture medium in order to create an FBS gradient. The inventors plated 80,000 cells for migration assay and ended the experiment 5 h later. For invasion assay, the inventors plated 50,000 and stopped the experiment 24 h later. At the end of the experiment, the remaining cells in the upper side of the Transwell device were removed. Migrating and invading cells at the bottom side of the Transwell device were fixed and stained with crystal violet for 30 minutes and then counted in the central field (×2.5 objective, Zeiss Axioplan microscope, AxioCamERc 5s).

Protein Extracts, Immunoprecipitation and Western Blot Analysis.

Proteins from human ovarian tumors enriched in epithelial cancer cells (up to 73% in average, with a minimum percentage of 55%) were extracted using boiling lysis buffer (50 mM Tris pH 6.8, 2% SDS, 5% glycerol, 2mMDTT, 2.5 mM EDTA, 2.5 mM EGTA, 4 mM Na3VO4 and 20 mM NaF) supplemented with 2× Halt Phosphatase inhibitor (Perbio #78420) and complete EDTA-free protease inhibitor cocktail tablet (Roche #1836170). The protein extract was snap frozen in liquid nitrogen and stored at −80° C. Protein concentration was evaluated using BCA Protein Assay kit—Reducing Agent Compatible according to the manufacturer's instructions (Thermo scientific).

After overnight serum starvation (—FBS), SKOV3 cells and SKOV3 stable cell lines were stimulated with 10% serum for 15 minutes (+FBS). SKOV3 cell line was treated or not with 5 μM or 10 μM MAP3K8 kinase inhibitor as indicated (KI_5 μM or KI_10 μM) for 1 h prior to serum stimulation. After treatment, cells were washed once with cold PBS and scraped on ice with lysis buffer (50 mM Tris-HCl pH 7.5, 1 mM EDTA, 1 mM EGTA, 1% triton X-100, 1 mM Na3VO4, 50mMNaF, 0.27M sucrose, 0.1% β-mercaptoethanol) supplemented with EDTA-free protease inhibitor cocktail tablet (Roche #1836170). For immunoprecipitation (IP), 4×106 SKOV3 cells were plated into 10 cm petri dish. 6 h later cells were transfected with increasing amount (2-6 μg) of N-terminal myc-tagged human MAP3K8 pcDNA3.1(+) plasmid DNA (Source BioScience imaGenes) using JetPrime transfection reagent according to the manufacturer's instructions with 1:4 ratio (Polyplus Transfection™) or with empty vector (Ctrl). 48 h post transfection, cells were washed with cold PBS and scraped on ice with IP lysis buffer (50 mM Tris-HCl pH 7.5, 150mMNaCl, 1 mM EDTA, 1 mM EGTA, 1% triton X-100, 1 mM Na3VO4, 10 mM β-glycerophosphate, 50mMNaF, 5 mM sodium pyrophosphate, 0.27M sucrose, 0.1% β-mercaptoethanol) supplemented with EDTA-free protease inhibitor cocktail tablet (Roche #1836170). Cells extracts were centrifuged at 13 000×g for 10 minutes at 4° C. The protein concentration of the supernatant was determined using the Bio-Rad Dc Protein Assay Kit according to the manufacturer's instructions (Bio-Rad Laboratories). After 10 minutes incubation on ice, protein lysates were spin down at 13,000 rpm and the supernatants were transferred into fresh tube. For IP 500 m of protein extract were processed immediately and incubated on a wheel overnight at 4° C., with 33 μl of myc antibody (9E10) coupled to magnetic beads (Dynabeads antibody coupling kit #1143.11D, Invitrogen) at 30 μg antibody per mg dynabeads. Beads were then washed 3 times using IP lysis buffer. Lastly, 20 μl of samples buffer 2× were added on top of the beads and boiled for 5 min at 95° C.

For western blot analysis, samples were loaded onto homemade or precast 10% polyacrylamide gels (Invitrogen). After electrophoresis, the proteins were transferred to a 0.45 μM PVDF transfer membrane (Immobilon-P, Millipore). Membranes were then blotted overnight at 4° C. with the appropriate primary antibodies: MAP3K8 (SantaCruz #sc-720), Phospho-MAP3K8 (T290) (Invitrogen #441370), GAPDH (Millipore #MAB374), Actin (Sigma #A5441) and Myc (9E10) (Roche #11667149001) or all the following antibodies from Cell Signalling Technology, Phospho-MAP3K8 (S400) (#4491), Phospho-MEK (#9121), MEK (#9126), Phospho-ERK (#9106), ERK (#9102), Phospho-JNK (#4668), JNK (#9258), Phospho-NF-κB (#3033), NF-κB (#8242), Phospho-p38 (#4511), p38 (#9218), Phospho-FAK (Tyr397) (#3283), FAK (#3285), p27kip1 (#2552), Cyclin D1 (#2922) and Rb (#9313). Specific binding of antibodies was detected using appropriate peroxidase-conjugated secondary antibodies (Jackson ImmunoResearch Laboratories #115-035-003 and 115-035-045), and was visualized by enhanced chemiluminescence detection (Western Lightning® Plus-ECL, PerkinElmer). Densitometric analyses of immunoblots were performed using ImageJ software.

Human Phospho-Kinase Array

The phosphorylation profile of 46 protein kinases was performed on protein extracts from SKOV3 cell line using the Proteome profiler antibody array according to the manufacturer's instructions (R&D systems #ARY003B). After overnight starvation, SKOV3 cells were stimulated with 10% serum for 30 minutes (FBS) or treated for 1 hour with 5 μM MAP3K8 kinase inhibitor prior to serum stimulation (FBS+KI).

Reverse Phase Protein Array (RPPA)

The Reverse Phase Protein Array has been performed at the RPPA platform of the Curie Institute following experimental procedures previously described (Troncale S. et al., PLoS one 2012). Briefly, serial diluted lysates were deposited onto nitrocellulose-covered slides, probed with primary antibodies and revealed with HRP-coupled secondary antibodies. Arrays were finally probed with Cy5-Streptavidin, dried and scanned using a GenePix 4000B microarray scanner (Molecular Devices). Spot intensity was determined with MicroVigene software (VigeneTech Inc.) data were normalized by Sypro Ruby protein stain. The antibodies used were all from Cell Signalling Technology: Phospho-NF-κB (#3033), NF-κB (#4764), Phospho-p38 (#4511), p38 (#9212).

Xenograft Experiments

Grafting experiments were performed by subcutaneous injection of 2.7×106 exponentially growing SKOV3 stable cell lines into each flank of 6-week-old female Swiss nude mice (at least five mice per group). Patient derived xenograft (PDX) models of EOC tumors were established at the Curie Institute with patient consent (Paris, France). For efficacy studies in PDX models, at least 8 mice per group were treated per os 5 times a week for 4 weeks either left untreated (Ctrl) or treated with the following MEK inhibitors AZD6244 at 50 mg/kg, (Selleckchem #S100817) or MEK162 25 mg/kg (Active Biochem #A-1128) both diluted into 2% DMSO, 10% 2-Hydroxypropyl-beta-cyclodextrin, 5% glucose in PBS. Tumor growth was evaluated by measuring two perpendicular diameters of tumors with a caliper twice a week. Individual tumor volumes were calculated as (V)=a×b2/2, with a being the major diameter, and b the minor one. For each tumor, V was reported to the initial volume as relative tumor volume (RTV). Means (and s.e.m) of RTV in the same treatment group were calculated, and growth curves were established as a function of time. The tumor growth inhibition (TGI) was calculated using the following formula: 100−100×(RTV from control mice/RTV from MEK inhibitor treated mice). Curie Institute ethical committee approved all experiments.

RT-qPCR for Human Alu Sequences on Lung Extracts

For lung metastasis detection in patient-derived tumor xenografts, the real-time PCR allows the detection of human RNA from mouse tissues. The presence of human cells within the host organ was quantified by mean of the transcript of human genes highly and exclusively represented in the human genome (Alu sequences). This method applied to the xenograft models greatly enhances the sensitivity of detection of invading human cells within lung tissues. Alu transcripts were considered to be detectable and quantifiable when the Ct value was below 35, and not detectable when the Ct value was above 35. The primers used for detection of Alu sequence were the following: 5′-TCACACCTGTAATCCCAGCACTTT-3′ (SEQ ID N^{o} 1) and 5′-GCCCAGGCTGGAGTGCAGT-3′ (SEQ ID N^{o} 2).

Statistical Analysis

To evaluate the prognostic value of the MAP3K8 protein levels, the inventors estimated, using the Kaplan-Meier method, the probabilities of overall survival (OS) and progression-free survival (PFS). The Kaplan-Meier curves were compared using the log-rank test. Iterative Kaplan-Meier analyses were performed to find optimal thresholds that maximally discriminate low-MAP3K8 and high-MAP3K8 groups. The edges of box-plots represent the 25th and the 75th percentiles, the solid line in the box the median value, and the error bar the 90th and 10th percentiles. Differences were considered to be statistically significant at values of p≦0.05. Graphs generally represent means±s.e.m obtained from independent experiments using adapted statistical test, as mentioned. The horizontal dark line on the scatter plots represents the median and the error bars the s.e.m. Spearman's correlation test was used to evaluate the correlation coefficient between 2 parameters. All statistical analyses were performed using R or Prism software.

Results

MAP3K8 Accumulation Impacts Patient Survival in High-Grade EOC

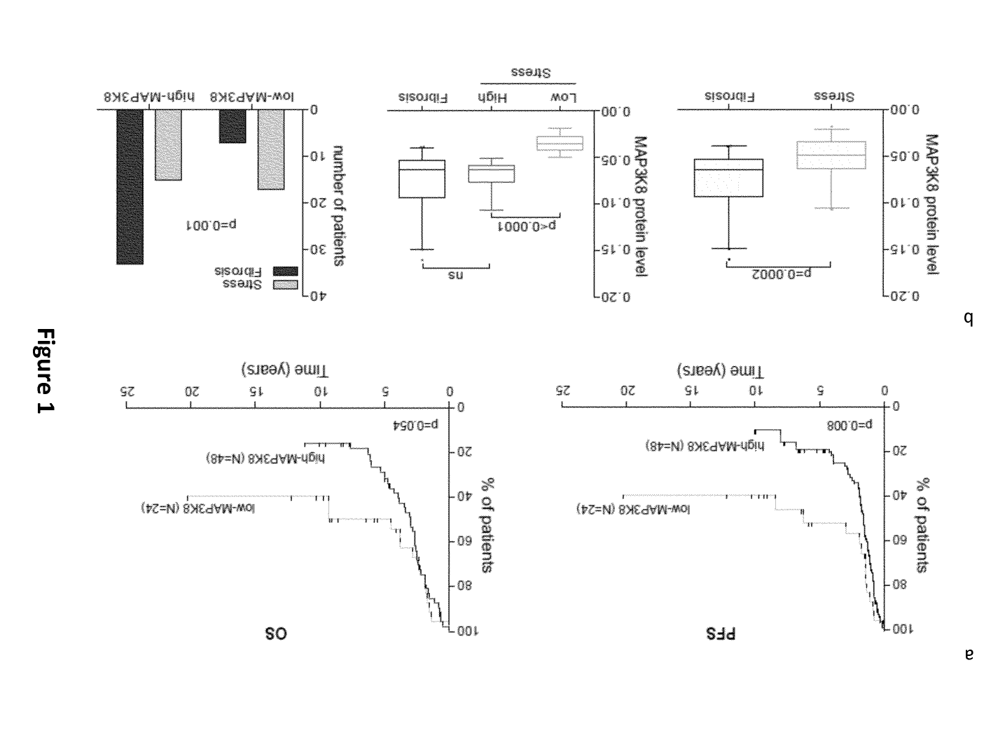

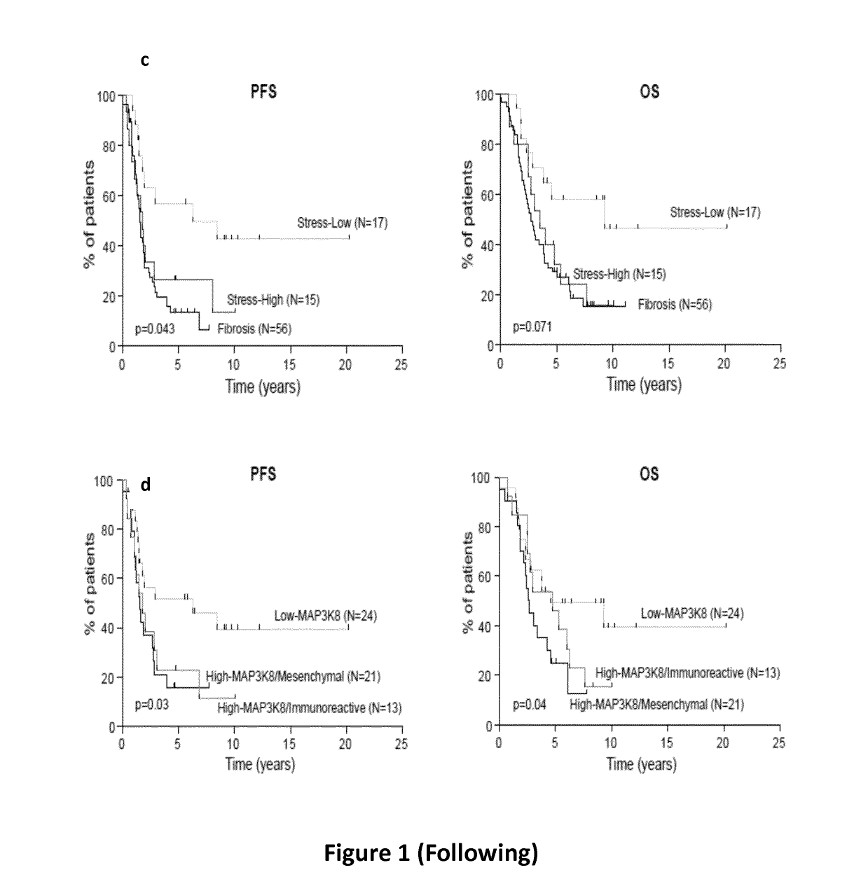

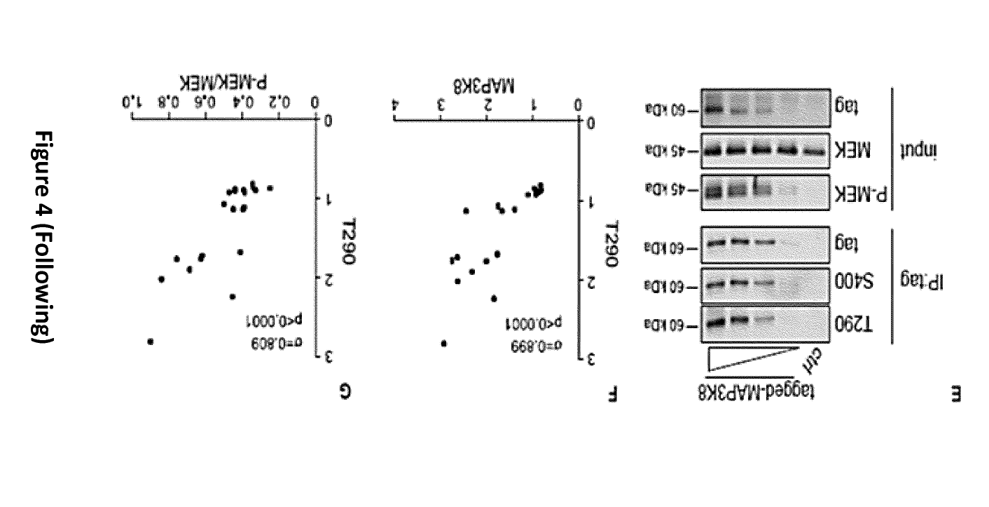

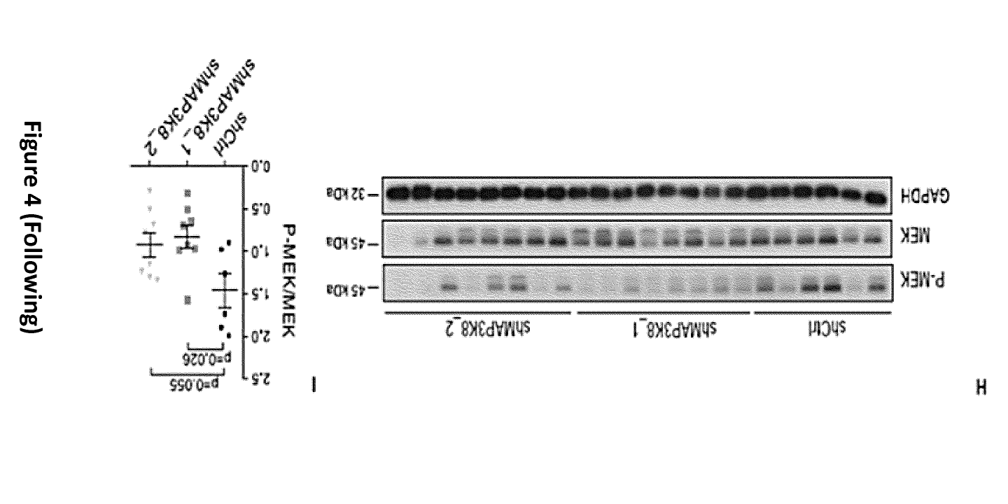

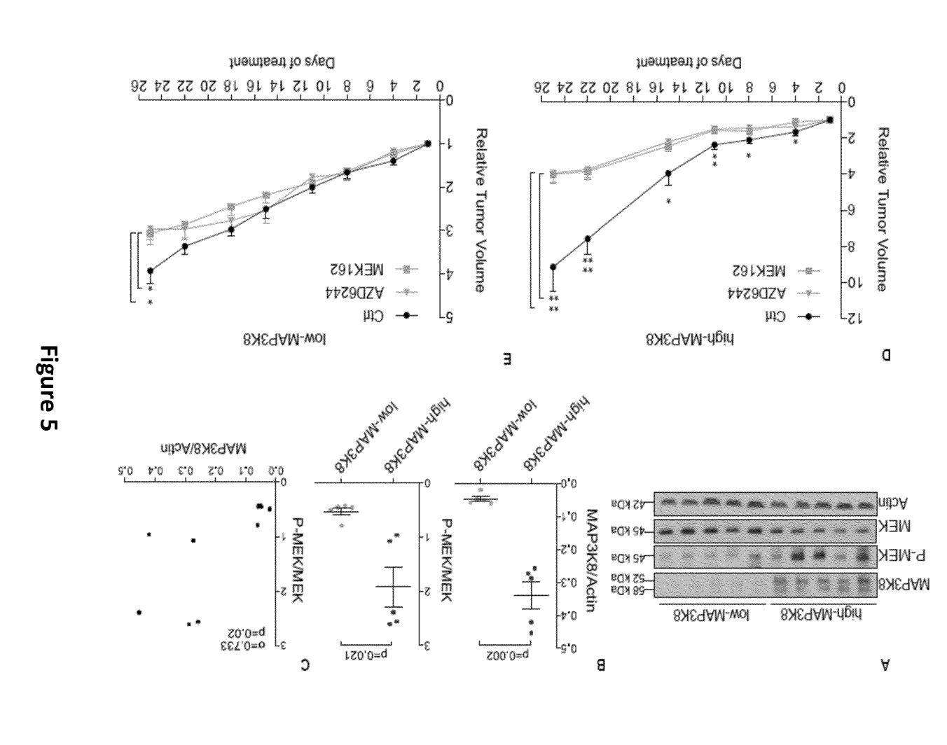

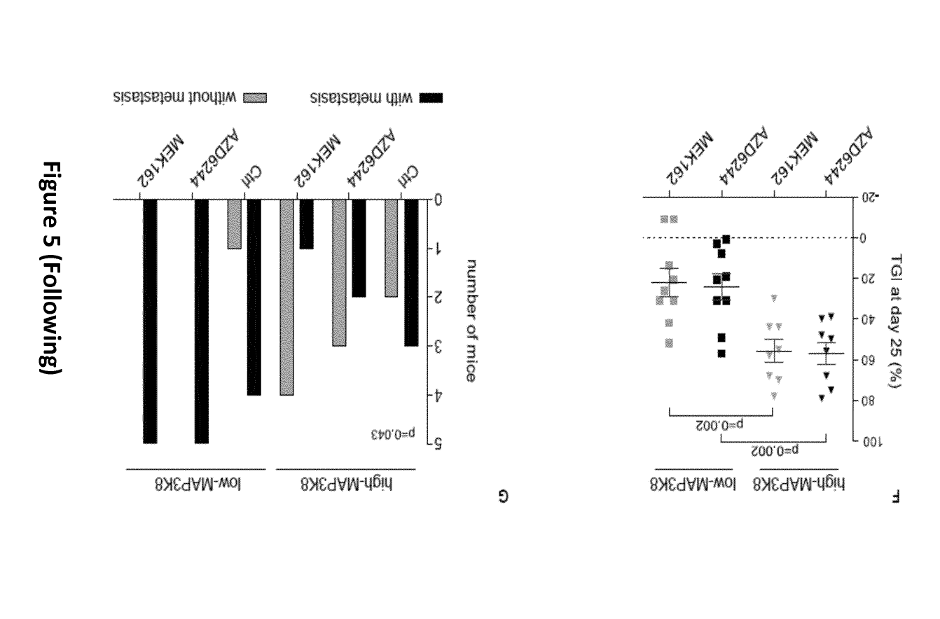

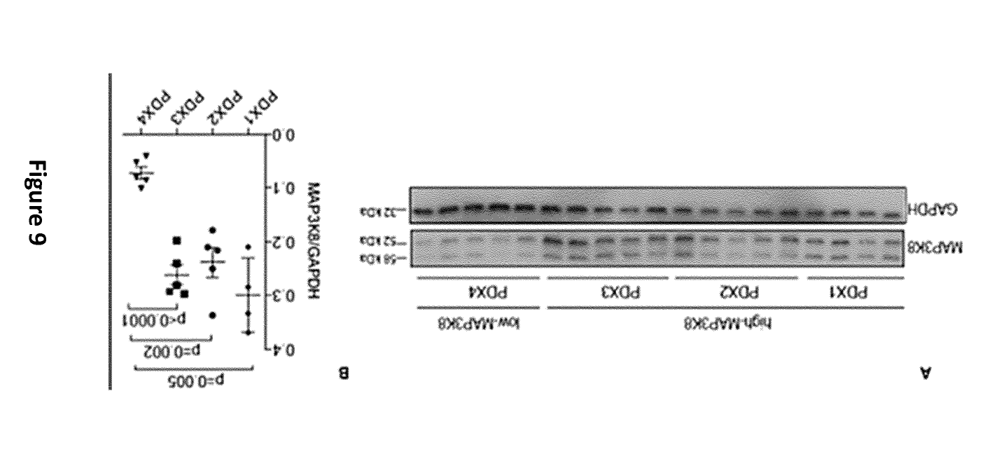

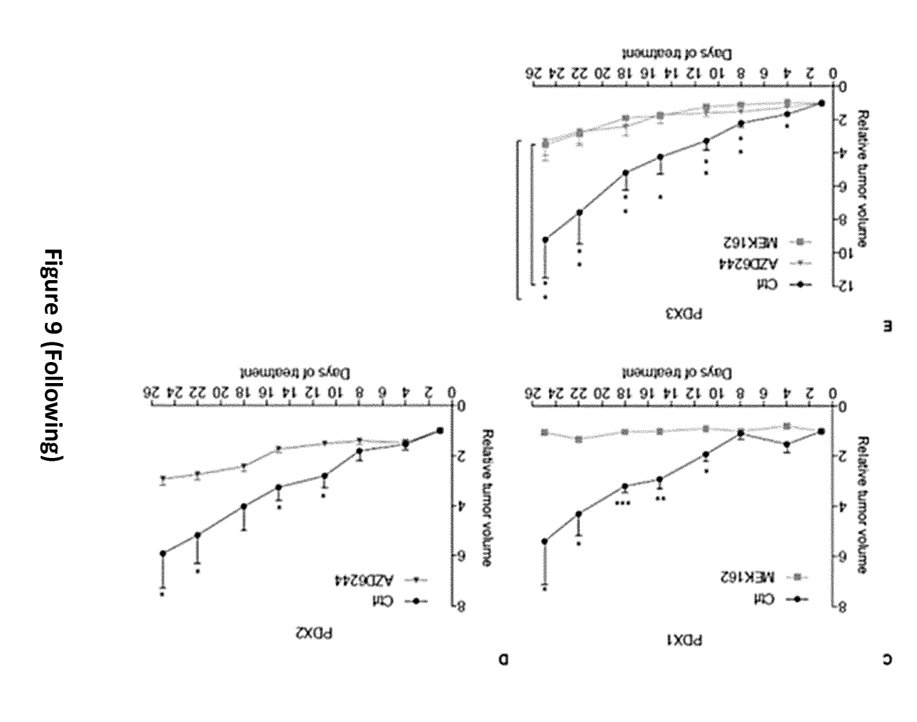

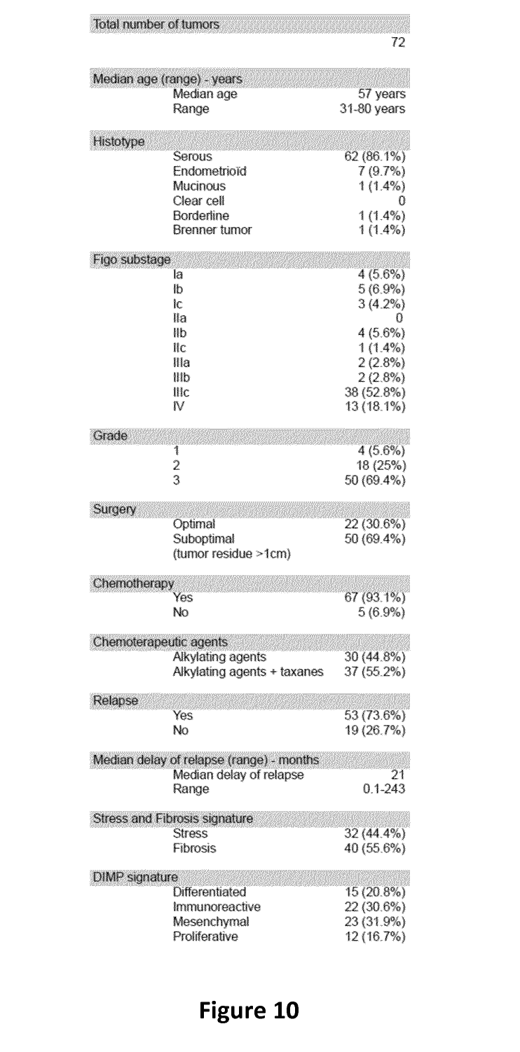

There is clear evidence of compensatory mechanisms between two main MAP3K, namely BRAF and MAP3K8, in regulating downstream MEK/ERK signaling pathway (Johannessen C. M. et al., Nature 2010). The inventors thus investigated the role of MAP3K8 in human high-grade serous EOC, in which BRAF mutations have been shown to be extremely rare (TCGA, Nature 2011). The inventors first tested the impact of MAP3K8 protein levels on patient survival (FIG. 1a and FIG. 7), using a set of ovarian adenocarcinomas including mostly high-grade tumors of serous histological subtype (later referred to as Curie Institute cohort, (Mateescu B. et al., Nat Med 2011), see also FIG. 10 for clinical details and patient information). MAP3K8 protein levels were assessed by western blot analysis performed on a large set of EOC samples, enriched in epithelial cancer cells with an average of 73% (FIG. 7 a). The inventors confirmed by immunohistochemistry that EOC patients could be classified into two subgroups characterized either by low- or high-MAP3K8 protein levels in epithelial cells (FIG. 7 b). Iterative Kaplan-Meier analyses allowed the inventors to find optimal thresholds that maximally discriminate subgroups of EOC patients for which tumors were characterized either by low- or high-MAP3K8 protein levels (FIG. 1). Both progression-free survival (PFS) (FIG. 1a, left panel) and overall survival (OS) (FIG. 1a, right panel) were markedly shortened in patients, whose tumors exhibited high-MAP3K8 protein levels, representing 75% of total patients. MAP3K8 prognostic value was only detected at protein levels, and not observed at mRNA levels, most probably because MAP3K8 mRNA and protein levels did not correlate in high-grade EOCs (data not shown). Clinical data analysis showed EOC patients with high-MAP3K8 protein levels were often characterized by a partial debulking status, a high grade and an advanced stage (FIG. 6). Thus the results of the inventors showed MAP3K8 protein accumulation is of poor prognosis.

The inventors next addressed the role of MAP3K8 in ovarian tumorigenesis in the context of the recent identification of EOC molecular subtypes, including “S&F” or “DIMP” signatures (Batista L. et al., Int J Biochem Cell Biol 2013; Mateescu B. et al., Nat Med 2011; TCGA, Nature 2011; Tothill R. W. et al., Clin Cancer Res 2008; Verhaak R. G. et al., J Clin Invest 2013). It is important to note all tumors of the “Mesenchymal” subtype defined by the “DIMP” signature correspond to the “Fibrosis” tumors, while the “Stress” signature brings together the “Differentiated”, “Immunoreactive” and “Proliferative” molecular subtypes. The inventors first investigated the distribution of the low- and high-MAP3K8 subgroups of patients in the context of the “S&F” signatures. “Fibrosis” tumors exhibited higher MAP3K8 protein levels than the “Stress” tumors (FIG. 1b, left panel and FIG. 7 a, b). Interestingly, the inventors were able to characterize two subgroups of tumors within the “Stress” molecular subtype according to MAP3K8 protein levels, hereafter named “Stress low-MAP3K8” and “Stress high-MAP3K8” (FIG. 1b, middle panel; FIG. 7 b). Indeed, MAP3K8 accumulated not only in almost all “Fibrosis” tumors but also in half of the “Stress” EOC (FIG. 1b, right panel). This result was confirmed using the “DIMP” signature, as the inventors observed a significant accumulation of MAP3K8 protein in the “Mesenchymal” subgroup of tumors (not shown). Moreover, in addition to the “Mesenchymal” subgroup, the “Immunoreactive” subgroup was enriched in tumors with “Stress” signature and in EOC with high-MAP3K8 protein levels (Not shown), as expected according to the already reported role of MAP3K8 in immune response (Arthur and Ley, Nature reviews Immunology 2013; Dumitru C. D. et al., Cell 2000; Gantke T. et al., Cell Res 2011). Finally, the inventors analyzed the impact of MAP3K8 protein levels on patient survival regarding the “S&F” and “DIMP” signatures. The inventors observed a worse PFS (FIG. 1c, left panel) and OS (FIG. 1c, right panel) for the “Stress high-MAP3K8” or the “Fibrosis” patients both characterized by high-MAP3K8 protein levels than for the “Stress low-MAP3K8” patients. Similarly, patients of the “Mesenchymal” and “Immunoreactive” subtypes, characterized by high-MAP3K8 protein levels, showed shortened PFS and OS when compared to tumors with low-MAP3K8 protein levels (FIG. 1d). Taken together, these data indicate MAP3K8 is a new reliable marker affecting patient survival in high-grade EOC that might be an interesting therapeutic target for patients with tumors characterized by MAP3K8 protein accumulation.

MAP3K8 Controls Proliferation, Migration and Invasion of Ovarian Cancer Cells

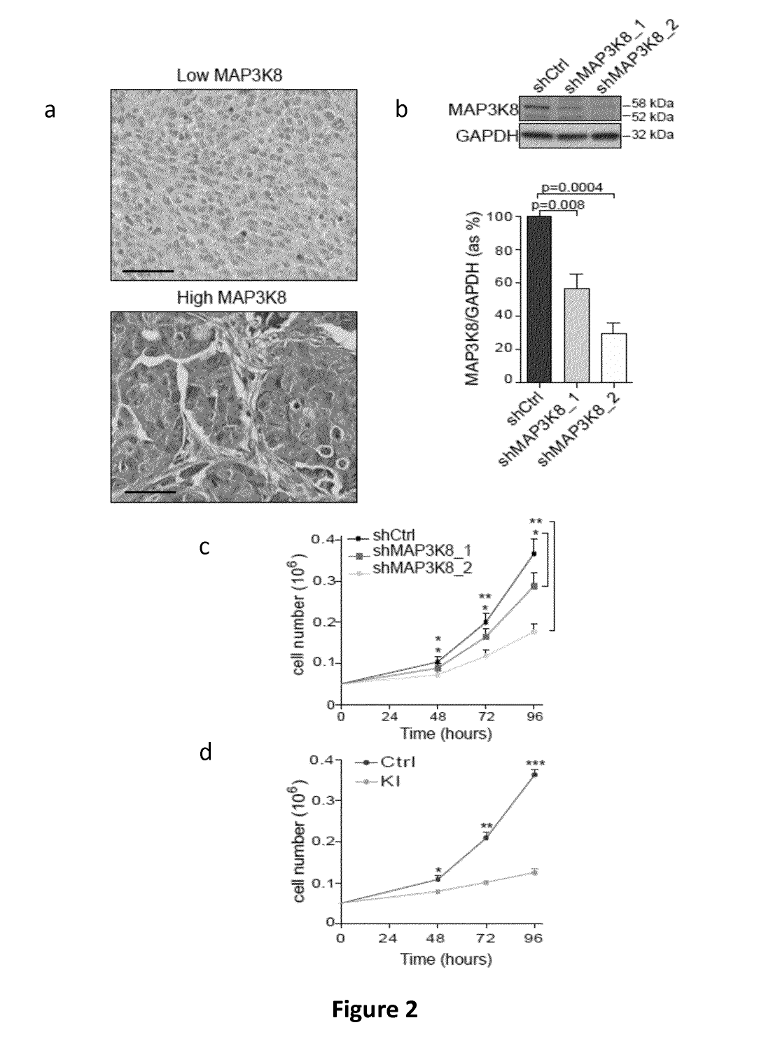

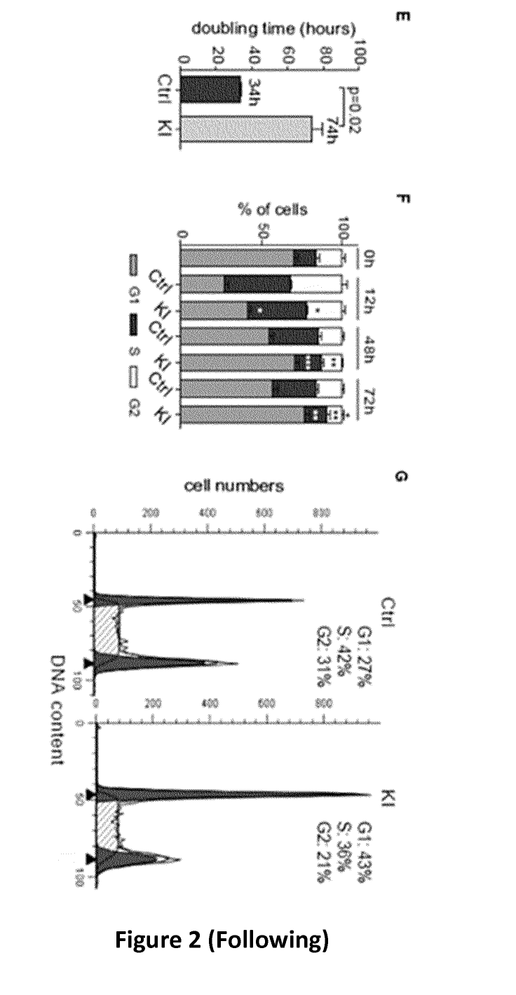

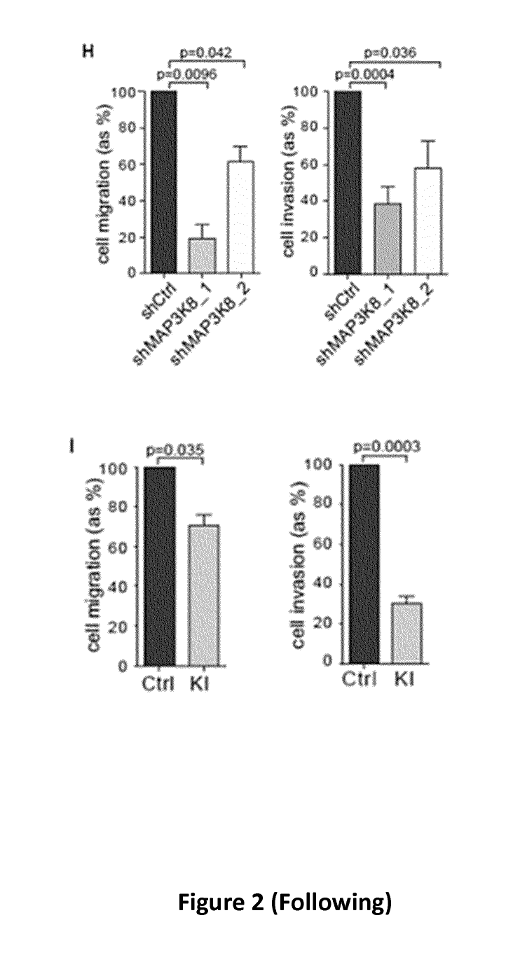

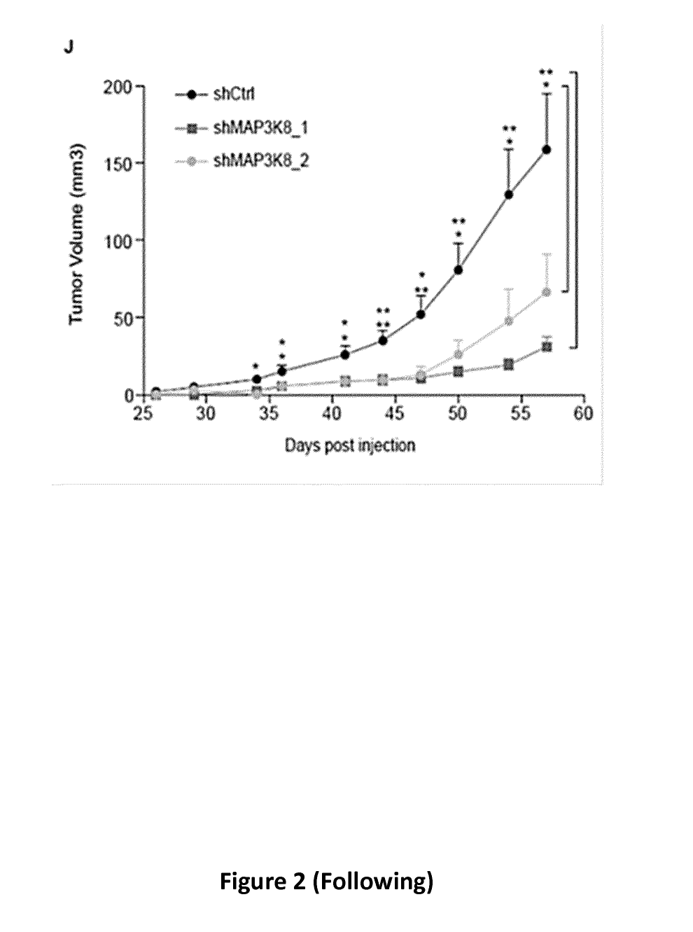

To investigate MAP3K8 function in ovarian tumorigenesis, the inventors analyzed MAP3K8 protein expression pattern by immunohistochemistry (IHC) in EOC. MAP3K8 is highly expressed in epithelial cancer cells (FIG. 2a), supporting this protein might have a cell-autonomous function. To test this hypothesis, the inventors stably inactivated MAP3K8 in SKOV3 ovarian cancer cells by expressing either non-targeting (shCtrl) or MAP3K8-targeting (shMAP3K8_1 and shMAP3K8_2) shRNAs (FIG. 2 b,c,h,j). The inventors confirmed a significant knockdown of MAP3K8 protein in shMAP3K8_1 and shMAP3K8_2 stable cell lines, (45% and 70% knockdown, respectively), as compared to shCtrl cells (FIG. 2b). The inventors also investigated the impact of MAP3K8 inhibition in ovarian cancer cells, using a specific ATP-competitive inhibitor of the MAP3K8 kinase (later referred to as KI, Calbiochem #616373), whose specificity and activity have been previously characterized and tested in depth (Kaila N. et al., Bioorganic & medicinal chemistry 2007) (FIG. 2d-g, i). The inventors first analyzed if MAP3K8 might be involved in controlling cancer cell proliferation. The inventors observed MAP3K8 knockdown in ovarian cancer cells reduced the total number of cells (FIG. 2c). This effect was confirmed using KI-treated cells (FIG. 2d). Interestingly, the inventors observed an increase in cell doubling time, following KI treatment (FIG. 2e), without any impact on cell viability (Not shown). These results suggest MAP3K8 inhibition might affect cell cycle regulation rather than cell death. The inventors thus performed cell cycle analysis using flow cytometry and observed a significant increase in the percentage of cells in G1 phase, as compared to untreated control cells (FIGS. 2f,g). These results indicate cells tend to accumulate in G1 phase upon MAP3K8 inhibition, therefore reducing their proliferation rate. To further determine if MAP3K8 might also affect ovarian tumor cell migration and/or invasion, the inventors performed transwell assay experiments. MAP3K8 inhibition by stable knockdown with shRNA (FIG. 2h, left panel) or KI treatment (FIG. 2i, left panel) significantly reduced cell migration. Cell invasion, assessed by invasion transwell biocoat assay, was also inhibited either upon MAP3K8 silencing (FIG. 2h, right panel) or KI treatment (FIG. 2i, right panel). Taken together, their data strongly support MAP3K8 regulates ovarian cancer cell proliferation, migration and invasion. It is important to note these data have been validated in three other ovarian cancer cell lines derived from mouse ovarian surface epithelial cells (MOSEC) (not shown) (Roby K. F. et al., Carcinogenesis 2000). Indeed, the inventors also observed a decrease in cell proliferation (not shown) and cell migration (not shown) upon MAP3K8 inhibition in MOSEC cell lines, further highlighting MAP3K8 cell autonomous functions in ovarian tumorigenesis is conserved amongst species. Finally, using mouse xenograft models, the inventors observed tumor growth was severely impaired following MAP3K8 depletion (shMAP3K8_1 and shMAP3K8_2) as compared to control tumors (shCtrl) (FIG. 2j), indicating MAP3K8 controls tumor growth in vivo. Thus, MAP3K8 regulates key features of ovarian cancer cells and promotes tumorigenesis in vivo, observations that explain, at least in part, MAP3K8 is associated with poor prognosis in EOC patients.

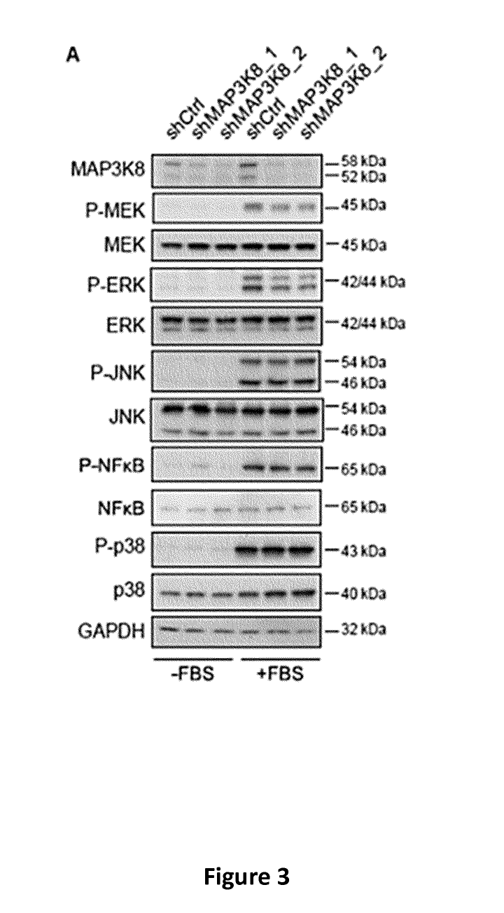

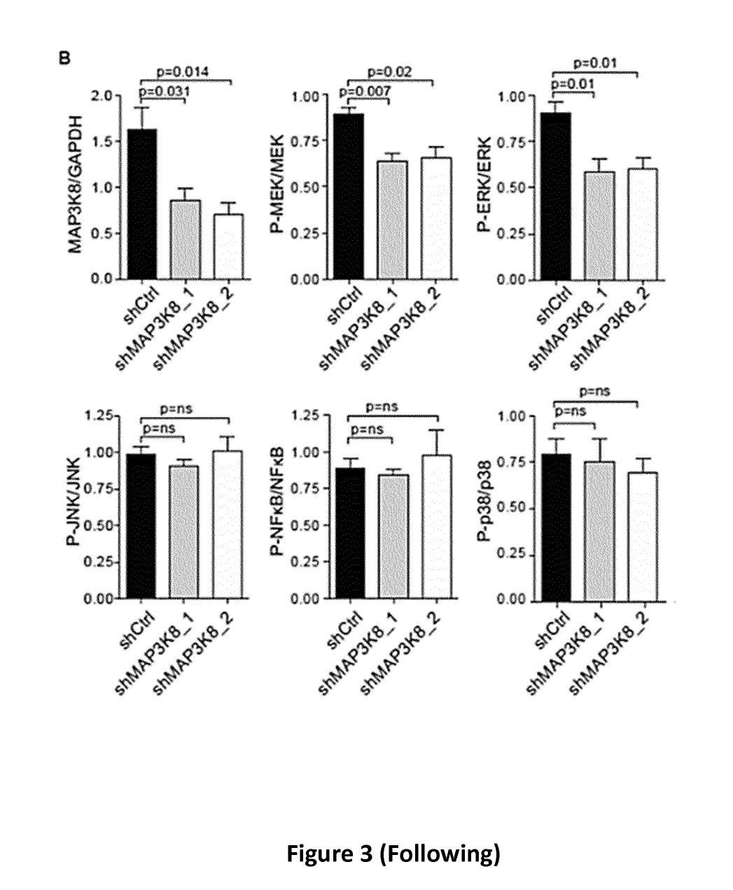

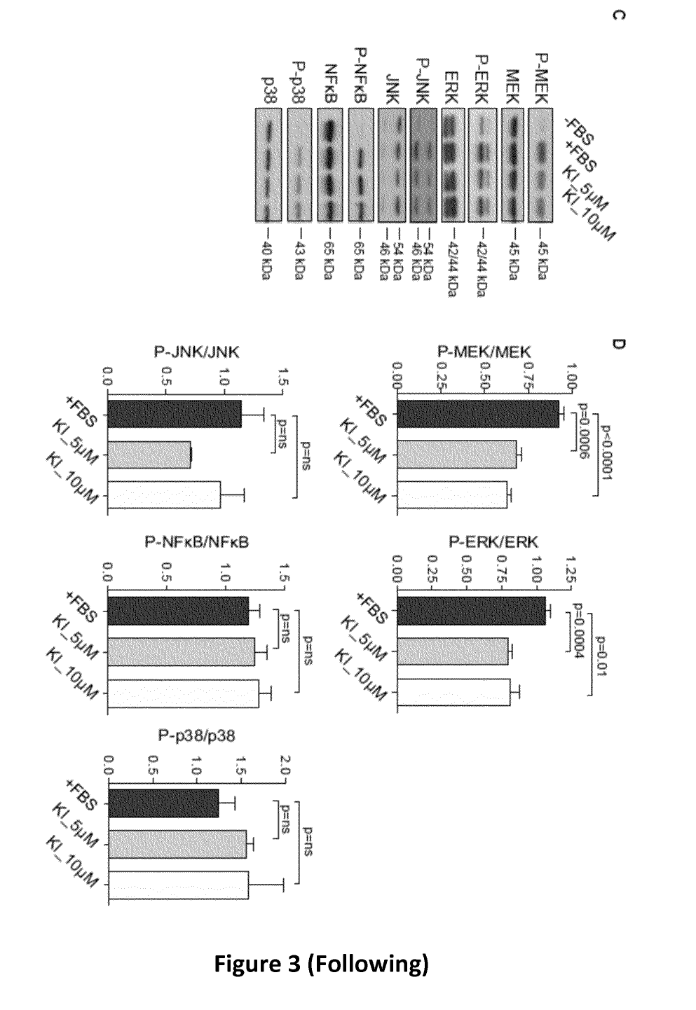

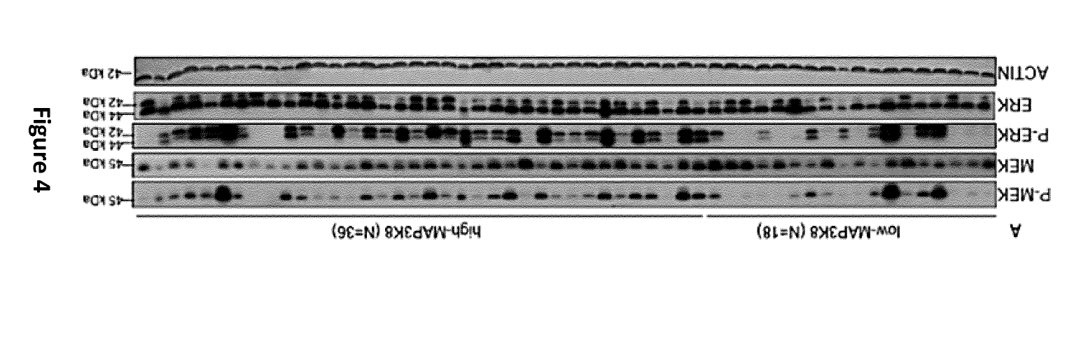

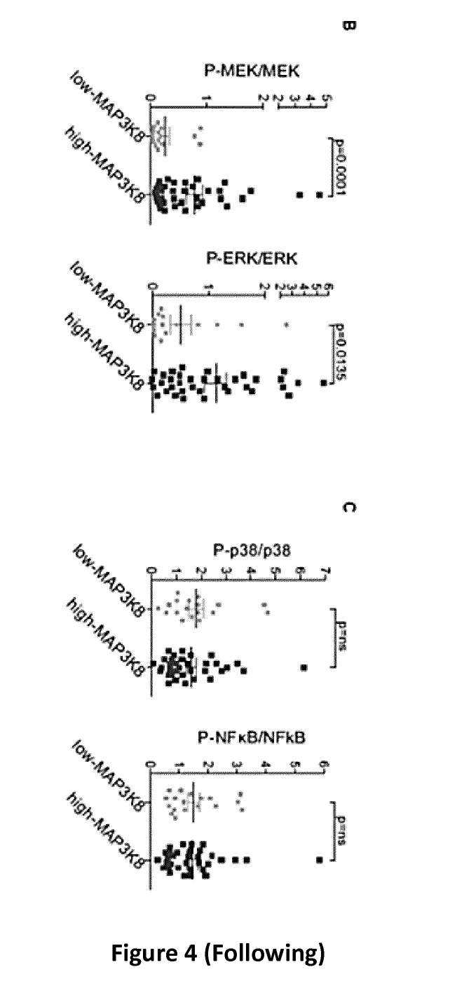

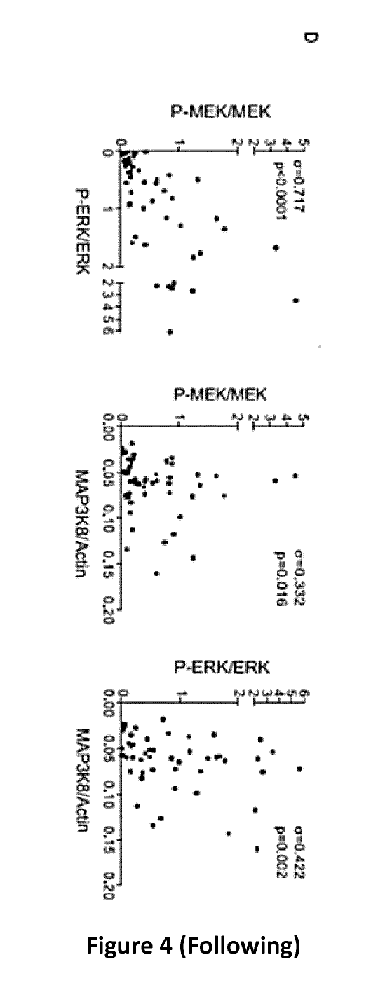

MAP3K8 is a significant readout for MEK/ERK signaling in EOC The inventors next looked at defining the signaling pathways involved. MAP3K8 downstream signaling is stimulus- and cell type-specific (Das S. et al., J Biol Chem 2005; Dumitru C. D. et al., Cell 2000; Gantke T. et al., Cell Res 2011). The MAPK pathways MEK/ERK, JNK and p38MAPK as well as NF-kB pathway are the main ones directly activated by MAP3K8 (Beinke S. et al., Mol Cell Biol 2003; Chiariello M. et al., Mol Cell Biol 2000; Jia Y. et al., Archives of biochemistry and biophysics 2005; Lin X. et al., Immunity 1999; Salmeron A. et al., EMBO 1996; Tsatsanis C. et al., Proc Natl Acad Sci USA 1998; Tsatsanis C. et al., Oncogene 1998; Waterfield M. R. et al., Mol Cell 2003). The inventors thus next investigated, the impact of MAP3K8 inhibition (by silencing and KI-treatment) on the above-mentioned pathways in ovarian cancer cells (FIG. 3). Serum-induced MEK and ERK phosphorylation was significantly reduced upon MAP3K8 knockdown as compared to control cells (FIG. 4 a,b, upper panels). In contrast, serum-induced p38MAPK and NF-κB phosphorylation was not affected by MAP3K8 silencing in ovarian cancer cells (FIG. 4 a,b, bottom panels). All these results were confirmed using KI treatment (FIG. 4 c,d). Regarding JNK phosphorylation, although not significant, the inventors tended to observe a slight decrease upon KI treatment (FIG. 4 c,d, bottom panel) but this potential effect was not confirmed when MAP3K8 was silenced (FIG. 4 a,b, bottom panel). Thus, MEK/ERK was the only common pathway inhibited using two complementary strategies impacting MAP3K8; suggesting MAP3K8 functions are mainly mediated through MEK/ERK kinases in ovarian cancer cells.

As the inventors observed MEK/ERK was the main pathway activated downstream MAP3K8 in ovarian cancer cells, the inventors intended to validate that observation in EOC patients. To do so, the inventors first tested whether MAP3K8 protein accumulation in EOC correlated with MEK/ERK activation (FIG. 4). The inventors stratified EOC samples as low- or high-MAP3K8 protein levels (as defined in FIG. 7). Tumors characterized by high-MAP3K8 protein levels exhibited a significant increase in both MEK and ERK activation, as compared to tumors with low-MAP3K8 protein levels (FIG. 4 a,b). Consistent with what the inventors observed in ovarian cancer cell lines, MAP3K8 protein level had no impact on p38MAPK and NF-κB pathways activation in human EOC (FIG. 4c). As expected, the inventors observed a significant positive correlation between MEK and ERK activation in EOC tumors (FIG. 4d, left panel). Importantly, the inventors also demonstrated MAP3K8 protein levels correlated with both MEK (FIG. 4d, middle panel) and ERK (FIG. 4d, right panel) activation in EOC, suggesting a predominant effect of MAP3K8 on MEK/ERK pathway in this pathology. To further demonstrate that the level of MAP3K8 protein correlates with its kinase activity and subsequent MEK/ERK activation, the inventors analyzed MAP3K8 phosphorylation state in ovarian cancer cells expressing increasing levels of MAP3K8 (FIG. 4e). Full catalytic activity of MAP3K8 has been previously shown to require its phosphorylation both at Threonine 290 (T290) and Serine 400 (S400) (Mieulet et al., Sci Signal 2010; Robinson M. J. et al., Mol Cell Biol 2007; Roget K. et al., Mol Cell Biol 2012; Stafford M. J. et al., FEBS Lett 2006). Increasing the expression of MAP3K8 in ovarian cancer cells was sufficient to induce MAP3K8 phosphorylation at both T290 and S400 phosphorylation sites, in a dose-dependent manner (FIG. 4e). Indeed, the inventors observed a clear correlation between MAP3K8 protein level and its phosphorylation state at both T290 and S400 phosphorylation sites (FIG. 40, indicating MAP3K8 protein level correlates with its kinase activity in ovarian cancer cells. Interestingly, MAP3K8 phosphorylation state also correlated with MEK activation (FIG. 4g), further highlighting MAP3K8 kinase could be a significant readout for MEK activation in ovarian cancers. Finally, in agreement with this assumption, the inventors observed MEK activation was significantly impaired in mouse ovarian tumors following MAP3K8-depletion (FIG. 5h,i), further indicating MAP3K8-dependent tumor growth is mediated by MEK activation in vivo. Taken together, their data show MAP3K8 protein levels correlate with its kinase activity and subsequent MEK/ERK activation, which participates to ovarian tumor growth.

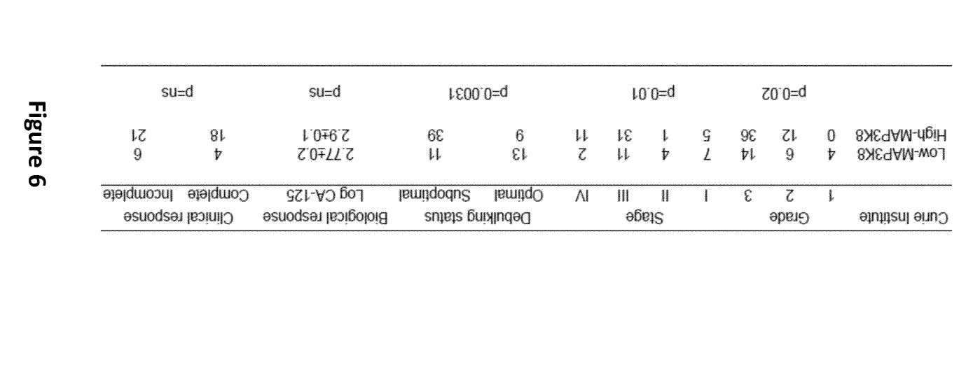

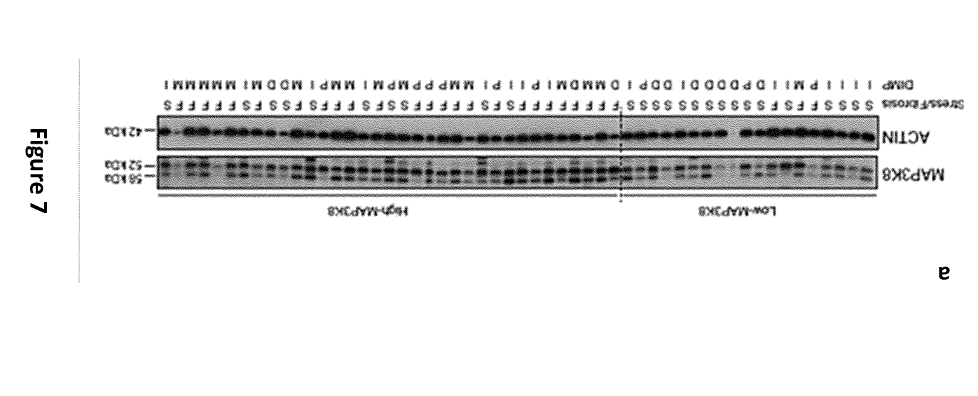

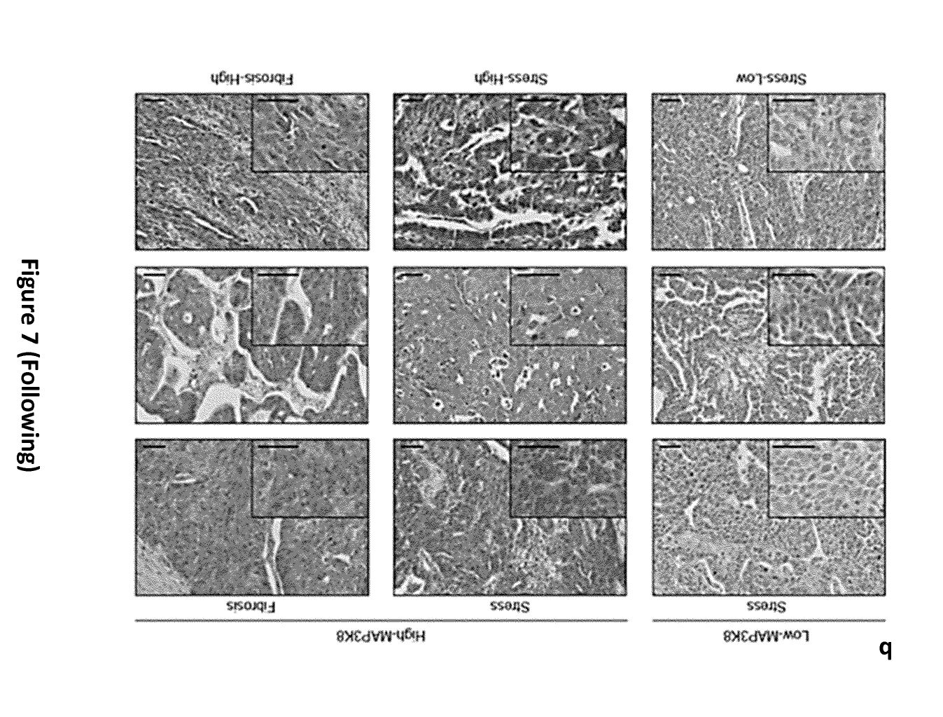

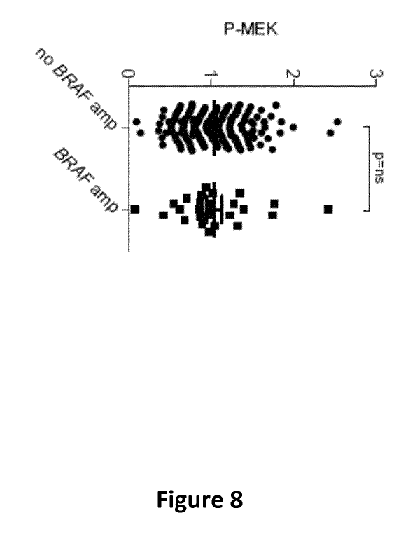

The correlation between MAP3K8 protein and MEK/ERK activation might be in part responsible for the poor prognosis observed in EOC patients harboring tumors with high-MAP3K8 protein levels (as shown above FIG. 1). The other well-known MAP3 kinase activating MEK/ERK pathway is BRAF. BRAF gene is often mutated in cancer, including low-grade EOC (Rahman M. A. et al., Experimental and molecular pathology 2013; Singer G. et al., J Natl Cancer Inst 2003). However, as previously mentioned, using publicly available datasets from a large cohort of high-grade serous EOC generated by TCGA (TCGA, Nature 2011), the inventors confirmed the percentage of BRAF mutation was no more than 1% in high-grade EOC. Still, in 11% cases the inventors detected genetic amplification of BRAF gene, but this was not correlated with MEK phosphorylation assessed by reverse phase protein array (RPPA) (FIG. 8) (TCGA, Nature 2011). Therefore it is unlikely BRAF would be involved in MEK/ERK pathway activation in high-grade EOC. Taken as a whole, these results strongly suggest MAP3K8 might be the sole MAP3 kinase involved in MEK/ERK pathway activation in high-grade EOC. This hypothesis is supported by their data revealing that MAP3K8 protein levels correlate with MEK/ERK activation in cells, mouse tumor models and human EOC.

MAP3K8 is a Predictive Marker for MEK Inhibitor Efficiency

As shown above, MEK, a direct MAP3K8 substrate, is constitutively active in high-grade EOC with high-MAP3K8 protein levels, further suggesting inhibiting MAP3K8/MEK pathway could be beneficial for these patients (75% of total high-grade EOC patients). Inhibiting MAP3K8 using KI was incompatible with in vivo experiments, due to cost reasons. Thus, to test their hypothesis, instead of inhibiting MAP3K8 using KI, the inventors opted for downstream MEK inhibition using 2 different ATP non-competitive MEK inhibitors, AZD6244/Selumetinib and MEK162, which are both tested in clinical trials for treatment of low-grade ovarian tumor patients (Farley J. et al., Lancet Oncol 2013; Study). The inventors thus tested these MEK inhibitors on patient-derived xenograft (PDX) models for high-grade EOC (FIG. 5 and FIG. 9). The inventors first verified that the PDX models, the inventors established from human primary EOC, reproduced their observations from patients. Indeed, the inventors identified 6 PDX models exhibiting either low- or high-MAP3K8 protein levels, two representatives ones being shown here (FIG. 5a, b left panel and FIG. 9a,b). As for human EOC, the inventors observed a much higher MEK activation in the different tumors derived from the PDX model with high-MAP3K8 protein levels than from the PDX model exhibiting low-MAP3K8 protein (FIG. 5a, b right panel). Similar to human EOC, the inventors detected a positive correlation between MAP3K8 protein levels and MEK activation in these PDX models (FIG. 5c). Consistent with the potential interest of the use of MEK inhibitors in EOC patients with high-MAP3K8 protein levels, the inventors observed treatment using MEK inhibitors markedly reduced tumor growth in high-MAP3K8 PDX model (FIG. 5d and FIG. 9 c-e), while it had much less impact on low-MAP3K8 PDX model (FIG. 5e). Indeed, both MEK inhibitors show almost 60% tumor growth inhibition (TGI) in high-MAP3K8 PDX model, while only 22% TGI was observed in low-MAP3K8 PDX model (FIG. 5f). Moreover, treatment with MEK inhibitors significantly reduced lung metastatic incidence in high-MAP3K8 PDX model, compared to low-MAP3K8 PDX model (FIG. 5g). Taken as a whole, their data demonstrate the potential interest for MEK inhibitors as a new therapeutic strategy in ovarian tumorigenesis and identify MAP3K8 as a new predictive marker for MEK inhibitors in human high-grade EOC.

### REFERENCES

Aparecida Alves, C., Silva, I. D., Villanova, F. E., Nicolau, S. M., Custodio, M. A., Bortoletto, C., and Goncalves, W. J. (2006). Differential gene expression profile reveals overexpression of MAP3K8 in invasive endometrioid carcinoma. Eur J Gynaecol Oncol 27, 589-593.
Arthur, J. S., and Ley, S. C. (2013). Mitogen-activated protein kinases in innate immunity. Nature reviews Immunology 13, 679-692.
Batista, L., Gruosso, T., and Mechta-Grigoriou, F. (2013). Ovarian cancer emerging subtypes: role of oxidative stress and fibrosis in tumour development and response to treatment. Int J Biochem Cell Biol 45, 1092-1098.
Beinke, S., Deka, J., Lang, V., Belich, M. P., Walker, P. A., Howell, S., Smerdon, S. J., Gamblin, S. J., and Ley, S. C. (2003). NF-kappaB1 p105 negatively regulates TPL-2 MEK kinase activity. Mol Cell Biol 23, 4739-4752.
Beinke, S., Robinson, M. J., Hugunin, M., and Ley, S. C. (2004). Lipopolysaccharide activation of the TPL-2/MEK/extracellular signal-regulated kinase mitogen-activated protein kinase cascade is regulated by IkappaB kinase-induced proteolysis of NF-kappaB1 p105. Mol Cell Biol 24, 9658-9667.
Belich, M. P., Salmeron, A., Johnston, L. H., and Ley, S. C. (1999). TPL-2 kinase regulates the proteolysis of the NF-kappaB-inhibitory protein NF-kappaB1 p105. Nature 397, 363-368.
Bendoraite A1, Knouf E C, Garg K S, Parkin R K, Kroh E M, O'Briant K C, Ventura A P, Godwin A K, Karlan B Y, Drescher C W, Urban N, Knudsen B S, Tewari M. Regulation of miR-200 family microRNAs and ZEB transcription factors in ovarian cancer: evidence supporting a mesothelial-to-epithelial transition. Gynecol Oncol. 2010 January; 116(1):117-25. doi: 10.1016/j.ygyno.2009.08.009. Epub 2009 Oct. 24.
Bentink, S., Haibe-Kains, B., Risch, T., Fan, J. B., Hirsch, M. S., Holton, K., Rubio, R., April, C., Chen, J., Wickham-Garcia, E., et al. Angiogenic mRNA and microRNA gene expression signature predicts a novel subtype of serous ovarian cancer. PLoS One 7, e30269.
Berman M L. Future directions in the surgical management of ovarian cancer.
Gynecol Oncol. 2003 August: 90(2 Pt 2):533-9. Carter, C. A., Misra, M., and Pelech, S. (2011). Proteomic analyses of lung lysates from short-term exposure of Fischer 344 rats to cigarette smoke. J Proteome Res 10, 3720-3731.
Chiariello, M., Marinissen, M. J., and Gutkind, J. S. (2000). Multiple mitogen-activated protein kinase signaling pathways connect the cot oncoprotein to the c-jun promoter and to cellular transformation. Mol Cell Biol 20, 1747-1758.
Chu, I. M., Hengst, L., and Slingerland, J. M. (2008). The Cdk inhibitor p27 in human cancer: prognostic potential and relevance to anticancer therapy. Nat Rev Cancer 8, 253-267.
Clark, A. M., Reynolds, S. H., Anderson, M., and Wiest, J. S. (2004). Mutational activation of the MAP3K8 protooncogene in lung cancer. Genes Chromosomes Cancer 41, 99-108.
Cronan, M. R., Nakamura, K., Johnson, N. L., Granger, D. A., Cuevas, B. D., Wang, J. G., Mackman, N., Scott, J. E., Dohlman, H. G., and Johnson, G. L. (2012). Defining MAP3 kinases required for MDA-MB-231 cell tumor growth and metastasis. Oncogene 31, 3889-3900.
Das, S., Cho, J., Lambertz, I., Kelliher, M. A., Eliopoulos, A. G., Du, K., and Tsichlis, P. N. (2005). Tpl2/cot signals activate ERK, JNK, and NF-kappaB in a cell-type and stimulus-specific manner. J Biol Chem 280, 23748-23757.
Davies, H., Bignell, G. R., Cox, C., Stephens, P., Edkins, S., Clegg, S., Teague, J., Woffendin, H., Garnett, M. J., Bottomley, W., et al. (2002). Mutations of the BRAF gene in human cancer. Nature 417, 949-954.
Decicco-Skinner, K. L., Trovato, E. L., Simmons, J. K., Lepage, P. K., and Wiest, J. S. (2011). Loss of tumor progression locus 2 (tp12) enhances tumorigenesis and inflammation in two-stage skin carcinogenesis. Oncogene 30, 389-397.
Diaz-Padilla, I., Malpica, A. L., Minig, L., Chiva, L. M., Gershenson, D. M., and Gonzalez-Martin, A. (2012). Ovarian low-grade serous carcinoma: a comprehensive update. Gynecol Oncol 126, 279-285.
Ding, L., Ellis, M. J., Li, S., Larson, D. E., Chen, K., Wallis, J. W., Harris, C. C., McLellan, M. D., Fulton, R. S., Fulton, L. L., et al. (2010). Genome remodelling in a basal-like breast cancer metastasis and xenograft. Nature 464, 999-1005.
Dumitru, C. D., Ceci, J. D., Tsatsanis, C., Kontoyiannis, D., Stamatakis, K., Lin, J. H., Patriotis, C., Jenkins, N. A., Copeland, N. G., Kollias, G., et al. (2000). TNF-alpha induction by LPS is regulated posttranscriptionally via a Tpl2/ERK-dependent pathway. Cell 103, 1071-1083.
Eliopoulos, A. G., Wang, C. C., Dumitru, C. D., and Tsichlis, P. N. (2003). Tpl2 transduces CD40 and TNF signals that activate ERK and regulates IgE induction by CD40. EMBO J 22, 3855-3864.
Farley, J., Brady, W. E., Vathipadiekal, V., Lankes, H. A., Coleman, R., Morgan, M. A., Mannel, R., Yamada, S. D., Mutch, D., Rodgers, W. H., et al. (2013). Selumetinib in women with recurrent low-grade serous carcinoma of the ovary or peritoneum: an open-label, single-arm, phase 2 study. Lancet Oncol 14, 134-140.
Fernandez, M., Manso, R., Bernaldo de Quiros, F., Lopez, P., Martin-Duce, A., and Alemany, S. (2011). Involvement of Cot activity in the proliferation of ALCL lymphoma cells. Biochem Biophys Res Commun 411, 655-660.
Gantke, T., Sriskantharajah, S., and Ley, S. C. (2011). Regulation and function of TPL-2, an IkappaB kinase-regulated MAP kinase kinase kinase. Cell Res 21, 131-145.
Gkirtzimanaki, K., Gkouskou, K. K., Oleksiewicz, U., Nikolaidis, G., Vyrla, D., Liontos, M., Pelekanou, V., Kanellis, D. C., Evangelou, K., Stathopoulos, E. N., et al. (2013). TPL2 kinase is a suppressor of lung carcinogenesis. Proc Natl Acad Sci USA 110, E1470-1479.
Handoyo, H., Stafford, M. J., McManus, E., Baltzis, D., Peggie, M., and Cohen, P. (2009). IRAK1-independent pathways required for the interleukin-1-stimulated activation of the Tpl2 catalytic subunit and its dissociation from ABIN2. Biochem J 424, 109-118.
Hatziapostolou, M., Koukos, G., Polytarchou, C., Kottakis, F., Serebrennikova, O., Kuliopulos, A., and Tsichlis, P. N. (2011). Tumor progression locus 2 mediates signal-induced increases in cytoplasmic calcium and cell migration. Sci Signal 4, ra55.
Hsu, C. Y., Bristow, R., Cha, M. S., Wang, B. G., Ho, C. L., Kurman, R. J., Wang, T. L., and Shih Ie, M. (2004). Characterization of active mitogen-activated protein kinase in ovarian serous carcinomas. Clin Cancer Res 10, 6432-6436.
Hu X1, Macdonald D M, Huettner P C, Feng Z, El Naga I M, Schwarz J K, Mutch D G, Grigsby P W, Pothe inventorsll S N, Wang X. A miR-200 microRNA cluster as prognostic marker in advanced ovarian cancer. Gynecol Oncol. 2009 September; 114(3):457-64. doi: 10.1016/j.ygyno.2009.05.022. Epub 2009 Jun. 5.
Hunger-Glaser, I., Salazar, E. P., Sinnett-Smith, J., and Rozengurt, E. (2003). Bombesin, lysophosphatidic acid, and epidermal growth factor rapidly stimulate focal adhesion kinase phosphorylation at Ser-910: requirement for ERK activation. J Biol Chem 278, 22631-22643.
Iorio MV1, Visone R, Di Leva G, Donati V, Petrocca F, Casalini P, Taccioli C, Volinia S, Liu C G, Alder H, Calin G A, Ménard S, Croce C M. MicroRNA signatures in human ovarian cancer. cancer Res 67, 8699-8707 (2007)
Jeong, J. H., Bhatia, A., Toth, Z., Oh, S., Inn, K. S., Liao, C. P., Roy-Burman, P., Melamed, J., Coetzee, G. A., and Jung, J. U. (2011). TPL2/COT/MAP3K8 (TPL2) activation promotes androgen depletion-independent (ADI) prostate cancer growth. PLoS One 6, e16205.
Jia, Y., Quinn, C. M., Bump, N. J., Clark, K. M., Clabbers, A., Hardman, J., Gagnon, A., Kamens, J., Tomlinson, M. J., Wishart, N., et al. (2005). Purification and kinetic characterization of recombinant human mitogen-activated protein kinase kinase kinase COT and the complexes with its cellular partner NF-kappa B1 p105. Archives of biochemistry and biophysics 441, 64-74.
Johannessen, C. M., Boehm, J. S., Kim, S. Y., Thomas, S. R., Wardwell, L., Johnson, L. A., Emery, C. M., Stransky, N., Cogdill, A. P., Barretina, J., et al. (2010). COT drives resistance to RAF inhibition through MAP kinase pathway reactivation. Nature 468, 968-972.
Kaila, N., Green, N., Li, H. Q., Hu, Y., Janz, K., Gavrin, L. K., Thomason, J., Tam, S., Powell, D., Cuozzo, J., et al. (2007). Identification of a novel class of selective Tpl2 kinase inhibitors: 4-Alkylamino-[1,7]naphthyridine-3-carbonitriles. Bioorganic & medicinal chemistry 15, 6425-6442.
Kaiser, F., Cook, D., Papoutsopoulou, S., Rajsbaum, R., Wu, X., Yang, H. T., Grant, S., Ricciardi-Castagnoli, P., Tsichlis, P. N., Ley, S. C., et al. (2009). TPL-2 negatively regulates interferon-beta production in macrophages and myeloid dendritic cells. The Journal of experimental medicine 206, 1863-1871.
Koliaraki, V., Roulis, M., and Kollias, G. (2012). Tpl2 regulates intestinal myofibroblast HGF release to suppress colitis-associated tumorigenesis. J Clin Invest 122, 4231-4242.
Kossatz, U., Vervoorts, J., Nickeleit, I., Sundberg, H. A., Arthur, J. S., Manns, M. P., and Malek, N. P. (2006). C-terminal phosphorylation controls the stability and function of p27kip1. EMBO J 25, 5159-5170.
Lang, V., Symons, A., Watton, S. J., Janzen, J., Soneji, Y., Beinke, S., Howell, S., and Ley, S. C. (2004). ABIN-2 forms a ternary complex with TPL-2 and NF-kappa B1 p105 and is essential for TPL-2 protein stability. Mol Cell Biol 24, 5235-5248.
Larrea, M. D., Liang, J., Da Silva, T., Hong, F., Shao, S. H., Han, K., Dumont, D., and Slingerland, J. M. (2008). Phosphorylation of p27Kip1 regulates assembly and activation of cyclin D1-Cdk4. Mol Cell Biol 28, 6462-6472.
Lee, H. W., Joo, K. M., Lim, J. E., Cho, H. J., Park, M. C., Seol, H. J., Seo, S. I., Lee, J. I., Kim, S., Jeong, B. C., et al. (2013). Tumor Progression Locus 2 (Tpl2) Kinase Contributes Tumor Growth and Metastasis of Clear Cell Renal Cell Carcinoma. Mol Cancer Res.
Lin, X., Cunningham, E. T., Jr., Mu, Y., Geleziunas, R., and Greene, W. C. (1999). The proto-oncogene Cot kinase participates in CD3/CD28 induction of NF-kappaB acting through the NF-kappaB-inducing kinase and IkappaB kinases. Immunity 10, 271-280.
Malpica, A., Deavers, M. T., Lu, K., Bodurka, D. C., Atkinson, E. N., Gershenson, D. M., and Silva, E. G. (2004). Grading ovarian serous carcinoma using a two-tier system. Am J Surg Pathol 28, 496-504.
Malpica, A., Deavers, M. T., Tomos, C., Kurman, R. J., Soslow, R., Seidman, J. D., Munsell, M. F., Gaertner, E., Frishberg, D., and Silva, E. G. (2007). Interobserver and intraobserver variability of a two-tier system for grading ovarian serous carcinoma. Am J Surg Pathol 31, 1168-1174.
Mateescu, B., Batista, L., Cardon, M., Gruosso, T., de Feraudy, Y., Mariani, O., Nicolas, A., Meyniel, J. P., Cottu, P., Sastre-Garau, X., et al. (2011). miR-141 and miR-200a act on ovarian tumorigenesis by controlling oxidative stress response. Nat Med 17, 1627-1635.
McLean, G. W., Carragher, N. O., Avizienyte, E., Evans, J., Brunton, V. G., and Frame, M. C. (2005). The role of focal-adhesion kinase in cancer—a new therapeutic opportunity. Nat Rev Cancer 5, 505-515.
Mieulet, V., Yan, L., Choisy, C., Sully, K., Procter, J., Kouroumalis, A., Krywawych, S., Pende, M., Ley, S. C., Moinard, C., et al. (2010). TPL-2-mediated activation of MAPK downstream of TLR4 signaling is coupled to arginine availability. Sci Signal 3, ra61.
Miller, C. R., Oliver, K. E., and Farley, J. H. (2014). MEK1/2 inhibitors in the treatment of gynecologic malignancies. Gynecol Oncol.
Milner, B. J., Allan, L. A., Eccles, D. M., Kitchener, H. C., Leonard, R. C., Kelly, K. F., Parkin, D. E., and Haites, N. E. (1993). p53 mutation is a common genetic event in ovarian carcinoma. Cancer Res 53, 2128-2132.
Mitra, S. K., Hanson, D. A., and Schlaepfer, D. D. (2005). Focal adhesion kinase: in command and control of cell motility. Nature reviews Molecular cell biology 6, 56-68.
Miyoshi, J., Higashi, T., Mukai, H., Ohuchi, T., and Kakunaga, T. (1991). Structure and transforming potential of the human cot oncogene encoding a putative protein kinase. Mol Cell Biol 11, 4088-4096.
Nam EJ1, Yoon H, Kim S W, Kim H, Kim Y T, Kim J H, Kim J W, Kim S. MicroRNA expression profiles in serous ovarian carcinoma. Clin Cancer Res. 2008 May 1; 14(9):2690-5. doi: 10.1158/1078-0432.CCR-07-1731.
Network, T. C. G. A. R. (2011). Integrated genomic analyses of ovarian carcinoma. Nature 474, 609-615.
Ohara, R., Hirota, S., Onoue, H., Nomura, S., Kitamura, Y., and Toyoshima, K. (1995). Identification of the cells expressing cot proto-oncogene mRNA. J Cell Sci 108 (Pt 1), 97-103.
Papoutsopoulou, S., Symons, A., Tharmalingham, T., Belich, M. P., Kaiser, F., Kioussis, D., O'Garra, A., Tybulewicz, V., and Ley, S. C. (2006). ABIN-2 is required for optimal activation of Erk MAP kinase in innate immune responses. Nat Immunol 7, 606-615.
Rahman, M. A., Salajegheh, A., Smith, R. A., and Lam, A. K. (2013). B-Raf mutation: a key player in molecular biology of cancer. Experimental and molecular pathology 95, 336-342.
Roberts, P. J., and Stinchcombe, T. E. (2013). KRAS mutation: should we test for it, and does it matter? Journal of clinical oncology: official journal of the American Society of Clinical Oncology 31, 1112-1121.
Robinson, M. J., Beinke, S., Kouroumalis, A., Tsichlis, P. N., and Ley, S. C. (2007). Phosphorylation of TPL-2 on serine 400 is essential for lipopolysaccharide activation of extracellular signal-regulated kinase in macrophages. Mol Cell Bio127, 7355-7364.
Roby, K. F., Taylor, C. C., Sweetwood, J. P., Cheng, Y., Pace, J. L., Tawfik, O., Persons, D. L., Smith, P. G., and Terranova, P. F. (2000). Development of a syngeneic mouse model for events related to ovarian cancer. Carcinogenesis 21, 585-591.
Roget, K., Ben-Addi, A., Mambole-Dema, A., Gantke, T., Yang, H. T., Janzen, J., Morrice, N., Abbott, D., and Ley, S. C. (2012). IkappaB kinase 2 regulates TPL-2 activation of extracellular signal-regulated kinases 1 and 2 by direct phosphorylation of TPL-2 serine 400. Mol Cell Biol 32, 4684-4690.
Rousseau, S., Papoutsopoulou, M., Symons, A., Cook, D., Lucocq, J. M., Prescott, A. R., O'Garra, A., Ley, S. C., and Cohen, P. (2008). TPL2-mediated activation of ERK1 and ERK2 regulates the processing of pre-TNF alpha in LPS-stimulated macrophages. J Cell Sci 121, 149-154.
Salmeron, A., Ahmad, T. B., Carlile, G. W., Pappin, D., Narsimhan, R. P., and Ley, S. C. (1996). Activation of MEK-1 and SEK-1 by Tp1-2 proto-oncoprotein, a novel MAP kinase kinase kinase. EMBO J 15, 817-826.
Schaller, M. D. (2010). Cellular functions of FAK kinases: insight into molecular mechanisms and novel functions. J Cell Sci 123, 1007-1013.
Schiappacassi, M., Lovisa, S., Lovat, F., Fabris, L., Colombatti, A., Belletti, B., and Baldassarre, G. (2011). Role of T198 modification in the regulation of p27(Kip1) protein stability and function. PLoS One 6, e17673.
Sebolt-Leopold, J. S., and Herrera, R. (2004). Targeting the mitogen-activated protein kinase cascade to treat cancer. Nat Rev Cancer 4, 937-947.
Serebrennikova, O. B., Tsatsanis, C., Mao, C., Gounaris, E., Ren, W., Siracusa, L. D., Eliopoulos, A. G., Khazaie, K., and Tsichlis, P. N. (2012). Tpl2 ablation promotes intestinal inflammation and tumorigenesis in Apcmin mice by inhibiting IL-10 secretion and regulatory T-cell generation. Proc Natl Acad Sci USA 109, E1082-1091.
Singer, G., Oldt, R., 3rd, Cohen, Y., Wang, B. G., Sidransky, D., Kurman, R. J., and Shih Ie, M. (2003). Mutations in BRAF and KRAS characterize the development of low-grade ovarian serous carcinoma. J Natl Cancer Inst 95, 484-486.
Singer, G., Stohr, R., Cope, L., Dehari, R., Hartmann, A., Cao, D. F., Wang, T. L., Kurman, R. J., and Shih Ie, M. (2005). Patterns of p53 mutations separate ovarian serous borderline tumors and low- and high-grade carcinomas and provide support for a new model of ovarian carcinogenesis: a mutational analysis with immunohistochemical correlation. Am J Surg Pathol 29, 218-224.
Solit, D. B., Garraway, L. A., Pratilas, C. A., Sawai, A., Getz, G., Basso, A., Ye, Q., Lobo, J. M., She, Y., Osman, I., et al. (2006). BRAF mutation predicts sensitivity to MEK inhibition. Nature 439, 358-362.
Sourvinos, G., Tsatsanis, C., and Spandidos, D. A. (1999). Overexpression of the Tpl-2/Cot oncogene in human breast cancer. Oncogene 18, 4968-4973.
Stafford, M. J., Morrice, N. A., Peggie, M. W., and Cohen, P. (2006). Interleukin-1 stimulated activation of the COT catalytic subunit through the phosphorylation of Thr290 and Ser62. FEBS Lett 580, 4010-4014.
Study, M. REF: NCT01849874 at ClinicalTrials.gov.
TCGA (2011). Integrated genomic analyses of ovarian carcinoma. Nature 474, 609-615.
Tothill, R. W., Tinker, A. V., George, J., Brown, R., Fox, S. B., Lade, S., Johnson, D. S., Trivett, M. K., Etemadmoghadam, D., Locandro, B., et al. (2008). Novel molecular subtypes of serous and endometrioid ovarian cancer linked to clinical outcome. Clin Cancer Res 14, 5198-5208.
Troncale, S., Barbet, A., Coulibaly, L., Henry, E., He, B., Barillot, E., Dubois, T., Hupe, P., and de Koning, L. (2012). NormaCurve: a SuperCurve-based method that simultaneously quantifies and normalizes reverse phase protein array data. PLoS One 7, e38686.
Tsatsanis, C., Patriotis, C., Bear, S. E., and Tsichlis, P. N. (1998a). The Tp1-2 protooncoprotein activates the nuclear factor of activated T cells and induces interleukin 2 expression in T cell lines. Proc Natl Acad Sci USA 95, 3827-3832.
Tsatsanis, C., Patriotis, C., and Tsichlis, P. N. (1998b). Tp1-2 induces IL-2 expression in T-cell lines by triggering multiple signaling pathways that activate NFAT and NF-kappaB. Oncogene 17, 2609-2618.
Tunca, B., Tezcan, G., Cecener, G., Egeli, U., Zorluoglu, A., Yilmazlar, T., Ak, S., Yerci, O., Ozturk, E., Umut, G., et al. (2013). Overexpression of CK20, MAP3K8 and EIFSA correlates with poor prognosis in early-onset colorectal cancer patients. J Cancer Res Clin Oncol 139, 691-702.
Vang, R., Shih Ie, M., and Kurman, R. J. (2009). Ovarian low-grade and high-grade serous carcinoma: pathogenesis, clinicopathologic and molecular biologic features, and diagnostic problems. Adv Anat Pathol 16, 267-282.
Verhaak, R. G., Tamayo, P., Yang, J. Y., Hubbard, D., Zhang, H., Creighton, C. J., Fereday, S., Lawrence, M., Carter, S. L., Mermel, C. H., et al. (2013). Prognostically relevant gene signatures of high-grade serous ovarian carcinoma. J Clin Invest 123, 517-525.
Vougioukalaki, M., Kanellis, D. C., Gkouskou, K., and Eliopoulos, A. G. (2011). Tpl2 kinase signal transduction in inflammation and cancer. Cancer Lett 304, 80-89.
Waterfield, M. R., Zhang, M., Norman, L. P., and Sun, S. C. (2003). NF-kappaB1/p105 regulates lipopolysaccharide-stimulated MAP kinase signaling by governing the stability and function of the Tpl2 kinase. Mol Cell 11, 685-694.
Wong, K. K., Tsang, Y. T., Deavers, M. T., Mok, S. C., Zu, Z., Sun, C., Malpica, A., Wolf, J. K., Lu, K. H., and Gershenson, D. M. (2010). BRAF mutation is rare in advanced-stage low-grade ovarian serous carcinomas. Am J Pathol 177, 1611-1617.
Yancik, R. (1993). Ovarian cancer. Age contrasts in incidence, histology, disease stage at diagnosis, and mortality. Cancer 71, 517-523.

1.-15. (canceled)

16. A method for treating ovarian cancer comprising
determining the expression level and/or activity level of MAP3K8 in a cancer sample from said patient;
administering a MEK inhibitor to said patient if the expression level and/or activity level of MAP3K8 is high.

17. The A method for treating ovarian cancer according to claim 16, wherein said ovarian cancer is an epithelial ovarian cancer.

18. The method for treating ovarian cancer according to claim 16, wherein said ovarian cancer is a high-grade and/or advanced-stage epithelial ovarian cancer.

19. The method for treating ovarian cancer according to claim 16, wherein said MEK inhibitor is selected from the group consisting of a small molecule, an antibody, a nucleic acid, an aptamer, a peptide, a polypeptide, a protein and any molecule preventing the interaction of MEK with a MEK interacting partner.

20. The method for treating ovarian cancer according to claim 16, wherein said MEK inhibitor is selected from a group consisting of a small molecule, an antibody against MEK and a nucleic acid molecule interfering specifically with MEK expression, such as an antisense against MEK, a siRNA against MEK and a shRNA against MEK.

21. The method for treating ovarian cancer according to claim 16, wherein said MEK inhibitor is a small molecule selected from the group consisting of artigenin, AS703026, AZD8330, AZD6244 (Selumetinib), BAY 869766, KT 5720, AS-252424, BIX 02189, Debromohymenialdisine, Hypothemycin, MEK Inhibiteur II, PD 0325901, PD 184,352, SB 203580, PD 184161, PD 198306, PD 98059, PD 318088, Selumetinib, SL-327, TAK-733, Trametinib, U-0126, U-0124, 2-Bromoaldisine, Myricetin, Chk2 Inhibiteur, Honokiol, cobimetinib, XL518, CI-1040 and MEK162.

22. The method for treating ovarian cancer according to claim 16, further comprising the step of treating said patient by surgery.

23. The method for treating ovarian cancer according to claim 16, further comprising the step of treating said patient by radiotherapy.

24. The method for treating ovarian cancer according to claim 16, further comprising the step of treating said patient by administering a chemotherapeutic agent.

25. The method for treating ovarian cancer according to claim 16, wherein the step of determining the expression level and/or activity level of MAP3K8 is carried out (i) by measuring the quantity of MAP3K8 protein by immunohistochemistry, semi-quantitative Western-Blot or protein or antibody arrays, and/or (ii) by measuring the activity of MAP3K8 by an enzymatic assay.

26. The method for treating ovarian cancer according to claim 16, wherein the method further comprises the step of comparing the expression level and/or the activity level of MAP3K8 to a reference expression level and/or a reference activity level.
